(19) United States
(12) Patent Application Publication
(10) Pub. No.: US 2003/0077616 A1
(43) Pub. Date: Apr. 24, 2003

(54) Biomolecule characterization using mass spectrometry and affinity tags

(75) Inventor: Lee O. Lomas, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Ciphergen Biosystems, Inc., Fremont, CA (US)

(21) Appl. No.: 10/125,819

(22) Filed: Apr. 18, 2002

Related U.S. Application Data
(60) Provisional application No. 60/285,630, filed on Apr. 19, 2001.

(30) Foreign Application Priority Data
Apr. 19, 2001 PCT/US02/12418

(51) Int. Cl. C12Q 1/68; G01N 33/53; G01N 33/543

(52) U.S. Cl. 435/6; 435/7.1; 436/518

(57) ABSTRACT

This invention relates to methods for comparing the relative amounts of biomolecules and identifying biomolecules in samples using affinity tags and mass spectrometry.

### CROSS-REFERENCES TO RELATED APPLICATIONS

This application claims the benefit of U.S. Provisional Patent Application No. 60/285,630, filed Apr. 19, 2001, the disclosure of which is incorporated herein by reference in its entirety for all purposes.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

With the advent of large-scale sequencing of the human genome and other genomes (e.g., Drosophila, C. elegans, E. coli, S. cerevisiae, Arabidopsis, Oryza sativa, etc.) it has been possible to rapidly identify genes that are expressed using arrays of oligonucleotide probes. Oligonucleotide probes can a priori be designed to bind to target nucleotides in a sample due to the nature of nucleic acid hydrogen bonding that occurs during hybridization. Arrays of oligonucleotide probes can be used to characterize the gene expression pattern of a sample and any changes in that expression pattern due to stimuli.

Similar methods and compositions are needed for large-scale, rapid analysis of protein and other biomolecule content of samples. Such methods and compositions will permit the characterization of the influence of agents on protein expression in cells and animals. Mass spectrometric methods in conjunction with protein and biomolecule purification techniques afford the ability to identify and assay for proteins that are present in a sample. However, purification often requires large amounts of starting material due to sample loss and the inefficiencies inherent in purification techniques. In addition, purification techniques often involve one or steps where the sample is eluted from a solid phase in order to be analyzed by mass spectrometry. Furthermore, purification techniques must often be individually tailored to a protein, and prior knowledge of the characteristics of a protein is often helpful in designing purification procedures. Thus, a need in the art exists for compositions that permit classes of proteins to be captured for subsequent mass spectrometric analysis. The present invention fulfills this and other needs in the art.

### SUMMARY OF THE INVENTION

This invention provides methods for determining the relative amounts of one or more biomolecules present in a first and a second sample, each containing two or more molecules. The biomolecule profiles of the first and second sample also overlap. In certain embodiments, the biomolecules are proteins. In some embodiments for determining the relative amounts of proteins, the methods involve contacting the first and second samples with an affinity tag having the formula A—R to generate one or more affinity tagged products. The affinity tagged products contain bonds between the affinity tag and a biomolecule. The group “A” of A—R contains an affinity label that specifically binds to a capture reagent, and the group “R” contains a biomolecule reactive group. The affinity tagged products are then immobilized in parallel on positionally distinguishable addresses on a substrate, containing the capture reagent bound thereto, to generate immobilized affinity tagged products. The amounts of affinity tagged products in the immobilized affinity tagged products are then determined using mass spectrometry. The mass spectrometry involves desorbing and ionizing the affinity tagged products from the immobilized affinity tagged products with an energy source and detecting the desorbed and ionized affinity tagged products with a detector. The amounts of affinity tagged products in the first and second samples are then compared to determine the relative amount of the protein present in the first and second samples.

In some embodiments, it is advantageous to contact the affinity tagged products with a polypeptide cleaving reagent. In some embodiments, the polypeptide cleaving reagent is a protease, such as chymotrypsin, trypsin, Endoproteinase Glu-C, Endoproteinase Asp-N, Endoproteinase Lys-C, Endoproteinase Arg-C, or Endoproteinase Arg-N. In other embodiments, the polypeptide cleaving reagent is cyanogen bromide or hydroxylamine

In another aspect of the invention, the methods involve a laser desorption-ionization mass spectrometer. In other embodiments, the laser desorption mass spectrometer is coupled to a quadrupole time-of-flight mass spectrometer.

In yet another aspect of the invention, a tandem mass spectrometer is used to carry out the methods of the present invention.

In certain embodiments of the present invention the affinity tag is a member selected from the group containing biotinyl-iodoacetylamidyl-4,7,10 trioxatridecanediamine; succinimidyl D-biotin; 6-((biotinoyl)amino)hexanoic acid, succinimidyl ester; 6-((biotinoyl)amino)hexanoic acid, sulfosuccinimidyl ester; 6-((6-((biotinoyl)amino)hexanoyl) amino)hexanoic acid, sulfosuccinimidyl ester; DNP-X-biocytin-X, succinimidyl ester; (1-biotinamide-4-[4′-(maleimidomethyl)cyclohexane-carboxamido]butane; (N-[6-(biotinamido)hexyl]-3′-(2′-pyridyldithio)propionamide; N-iodoacetyl-N-biotinylhexylenediamine; [+]-biotinyl-iodoacetamidyl-3,6-dioxaoctanediamine; N-(biotinoyl)-N′-(iodoacetyl)ethylenediamine; Nα-(3-maleimidylpropionyl)biocytin; cis-tetrahydro-2-oxothieno[3,4-d]-imidazoline-4-valeric acid hydrazide; biotin-LC-hydrazide biocytin hydrazide; and N-(4-azido-2-nitrophenyl)-aminopropyl-N′-(N-d-biotinyl-3-aminopropyl)-N′-methyl-1,3-propanediamine.

The present invention also provides methods for determining the identity of one or more proteins in a sample. In certain embodiments, the biomolecules are proteins. In some embodiments for determining the relative amounts of proteins, the methods involve contacting the sample with an affinity tag having the formula A—R to generate one or more affinity tagged products. The affinity tagged products contain bonds between the affinity tag and a biomolecule. The group “A” of A—R contains an affinity label that specifically binds to a capture reagent, and the group “R” contains a biomolecule reactive group. The affinity tagged products are immobilized on a substrate, containing the capture reagent bound thereto, to generate immobilized affinity tagged products. The identity of proteins in the affinity tagged products in the immobilized affinity tagged products are then determined using mass spectrometry. The mass spectrometry involves desorbing and ionizing the affinity tagged products from the immobilized affinity tagged products with an energy source and detecting the desorbed and ionized affinity tagged products with a detector.

In certain embodiments, the immobilized affinity tagged products are contacted with a polypeptide cleaving reagent to create polypeptide cleavage fragments. Mass spectrometry, involving a first and second mass spectrometer, is then used to determine the identity of one the proteins. The mass spectrometry involves desorbing the protein cleaving reagent fragments from the substrate-bound capture reagent to generate parent ion peptides. A parent ion peptide is then selected for subsequent fragmentation with a first mass spectrometer. The selected parent ion peptide is then fragmented under selected fragmentation conditions in the first mass spectrometer to generate product ion fragments. A first mass spectrum of the product ion fragments is then generated with a second mass spectrometer. A database is then accessed with a programmable digital computer. The database contains one or more predicted mass spectra of amino acid sequences. An algorithm is executed with a programmable digital computer to determine at least a first measure for each of the predicted mass spectra. The first measure is an indication of the closeness-of-fit between the first mass spectrum and each of the predicted mass spectra.

In yet another aspect, the present provides for methods for determining the mass of a biomolecule. These methods involve contacting the biomolecule with an affinity tag having the formula A—R to generate one or more affinity tagged products. A contains an affinity label that specifically binds to a capture reagent and R contains a protein reactive group. The R group reacts with a functional group on the biomolecules to generate affinity tagged products containing bonds between the affinity tag and the biomolecules. The affinity tagged products are then immobilized on a substrate, containing the capture reagent bound thereto, to generate immobilized affinity tagged products. The mass of the affinity tagged products are determined by mass spectrometry, which involves desorbing and ionizing the affinity tagged products from the immobilized affinity tagged products with an energy source and detecting the desorbed and ionized affinity tagged products with a detector.

These and other aspects of the present invention will become apparent upon reference to the following detailed description.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Analyte” refers to any component of a sample that is desired to be detected. The term can refer to a single component or a plurality of components in the sample.

“Eluant” refers to an agent, typically a solution, that is used to affect or modify adsorption of an analyte to an adsorbent of an adsorption surface. Eluants also are referred to herein as “selectivity threshold modifiers.”

“Elution characteristic” refers to a physical or chemical characteristic of an eluant that contributes to its ability to affect or modify adsorption of an analyte to an adsorbent of an adsorption surface. Two eluants have different elution characteristics if, when put in contact with an analyte and adsorbent, the degree of affinity of the analyte for the adsorbent differs. Elution characteristics include, for example, pH, ionic strength, degree of chaotropism, detergent strength, and temperature.

The phrase “determining the relative amounts” means measuring quantitatively or qualitatively the amounts of one or more analytes (e.g., proteins, biomolecules, etc.).

Two samples have “overlapping biomolecule profiles” when two or more biomolecules are present in each sample and when the samples have at least one biomolecule in common.

The term “biomolecules” refers to a molecule composed of one or more amino acid, nucleotide, carbohydrate, lipid, etc., such as proteins, polysaccharides, carbohydrates, lipids, nucleic acids, glycolipids, glycoproteins, lipoproteins, etc.

“Biopolymer” refers to a biomolecule in polymeric form, e.g., polypeptides, polynucleotides, polysaccharides and polyglycerides (e.g., di- or tri-glycerides).

The term “proteins” refers to amino acid polymers such as peptides, protein, polypeptides, etc. The terms “peptides,” “proteins,” and “polypeptides,” are considered interchangeable. The term “proteins” encompasses amino acid polymers that have been post-translationally modified with phosphoryl groups, carbohydrate groups, lipid groups, etc.

“Biologic sample” and “biological sample” identically refer to a sample derived from at least a portion of an organism capable of replication. As used herein, a biologic sample can be derived from any of the known taxonomic kingdoms, including virus, prokaryote, single celled eukaryote and multicellular eukaryote. The biologic sample can derive from the entirety of the organism or a portion thereof, including from a cultured portion thereof. Biologic samples can be in any physical form appropriate to the context, including homogenate, subcellular fractionate, lysate and fluid. “Complex biologic sample” refers to a biologic sample comprising at least 100 different protein species. A “moderately complex biologic sample” refers to a biologic sample comprising at least 20 different protein species.

“Small organic molecule” refers to organic molecules of a size comparable to those organic molecules generally used in pharmaceuticals. The term excludes organic biopolymers (e.g., proteins, nucleic acids, etc.). Small organic molecules as used herein typically range in size up to about 5000 Da, up to about 2500 Da, up to about 2000 Da, or up to about 1000 Da.

“Molecular binding partners”—and equivalently, “specific binding partners”—refer to pairs of molecules, typically pairs of biomolecules, that exhibit specific binding. Nonlimiting examples are receptor and ligand, antibody and antigen, biotin and avidin, and biotin and streptavidin.

“Receptor” refers to a molecule, typically a macromolecule, that can be found in, but need not necessarily have been derived from, a biologic sample, and that can participate in specific binding with a ligand. The term further includes fragments and derivatives that remain capable of specific ligand binding.

“Ligand” refers to any compound that can participate in specific binding with a designated receptor or antibody.

A “biomolecule reactive group” is a chemical entity or moiety that can form a bond (e.g., a covalent bond, a non-covalent bond, etc.) with functional groups (e.g., primary amines, secondary amines, hydroxyls, amines, imidazole rings, carboxylates, sulfhydryls, disulfides, thioethers, imidazolyls, phenol rings, indolyl rings, guanidinyl groups, vicinal diols, etc.), such as those that exist on biomolecules. These functional groups include, but are not limited to, proteins, amino acids, carbohydrates, nucleic acids, lipids, and other biomolecules. In addition, functional groups can be produced on proteins, amino acids, carbohydrates, nucleic acids, lipids, and other biomolecules by reacting them with a reagent (e.g., periodate reduction of vicinal diols). Examples of biomolecule reactive groups include, without limitation, an activated acyl group, an activated alkyl group, a pyridyl-disulfide group, a malemide group, an iodoacetamidyl group, an alkyl halide, an aryl halide, an sulfonyl halide, a nitrile, an α-haloacyl group, an epoxide, an oxirane, a diazonium group, a diazoalkane, a diacetyl group, succinimidyl ester, N-hydroxysuccinimidyl ester, a sulfosuccinimyl ester, an isothiocyanate, an isocyanate, a sulfonyl chloride, a dichlorotriazine, an acyl azide, a pentafluorophenyl ester, a tetrafluorophenyl ester, a 4-sulfo-2,3,5,6-tetrafluorophenyl ester, a hydrazide, a 5′-(4-Fluorosulfonylbenzoyl)adenosine, and a 5-p-fluorosulfonylbenzoyl guanosine.

The term “immobilizing” refers to affixing, attaching, or binding a first molecule to a surface or another molecule either directly or indirectly.

A “capture reagent” is a substance that can selectively bind to an affinity label.

The phrase “specifically (or selectively) binds to” in the context of an affinity label or affinity tag refers to a binding reaction in which the capture reagent binds to a molecule containing an affinity tag (e.g., an affinity tagged product, an affinity label, etc.). Typically, the capture reagent “specifically (or selectively) binds to” the affinity label portion of an affinity tag. Thus, under designated binding conditions, a specifically binding capture reagent will bind to a molecule containing the requisite affinity label and under the same designated binding conditions, the specifically binding capture reagent does not bind in a significant amount to a molecule that does not contain the requisite affinity label. Typically, a capture reagent “specifically” binds to a molecule containing an affinity label when the number of molecules containing an affinity label that are bound to the capture reagents is at least twice the background binding, more preferably 10-100 times background, observed using a molecule that does not contain an affinity label as a control.

A “polypeptide cleaving reagent” is any agent, compound, or substance that can be used to break a peptide bond or otherwise restrict a peptide into smaller units (i.e., “polypeptide cleavage fragments”) e.g., smaller peptides or amino acids. Examples of “polypeptide cleaving reagents” include, without limitation, proteases and chemicals. Examples of proteases include, chymotrypsin, trypsin, Endoproteinase Glu-C, Endoproteinase Asp-N, Endoproteinase Lys-C, Endoproteinase Arg-C, Endoproteinase Arg-N, Factor Xa protease, thrombin, enterokinase, V5 protease, and the tobacco etch virus protease. Examples of “polypeptide cleaving reagents” further include cyanogen bromide and hydroxylamine.

“Affinity tagged products” contain bonds between a biomolecule and an affinity label. The bonds can be covalent or non-covalent (e.g., ionic, hydrogen, etc.) bonds.

The phrase “in parallel” refers to carrying out a step on two or more different samples separately. The step can be carried out on the samples simultaneously, or non-simultaneously.

“Positionally distinguishable addresses” are separate areas on the same object or separate areas on separate objects that can be identified as to their place on the object(s).

A “biomolecule cleaving reagent” is any agent, compound, or substance that can be used to break a bond in a biomolecule or otherwise restrict a biomolecule into smaller units, biomolecule cleavage fragments, e.g., smaller peptides or amino acids, restriction fragments of nucleotides, smaller polysaccharides or carbohydrates, etc.

“Biomolecule cleavage fragments” are subsequences of a biomolecule that are produced through treatment of a biomolecule with a biomolecule cleaving reagent.

A “polysaccharide cleaving reagent” is any agent, compound, or substance that can be used to break a bond in a polysaccharide, restrict a polysaccharide into smaller units, polysaccharide cleavage fragments, e.g., smaller polysaccharides or carbohydrates, or cleave one or more carbohydrates from a biomolecule. Examples of “polysaccharide cleaving reagents” include glyocosidases, endoglycosidases and exoglycosideases.

A “DNA cleaving reagent” is any agent, compound, or substance that can be used to break a bond in a DNA molecule or otherwise restrict a DNA molecule into smaller units, DNA cleavage fragments, e.g., fragments of the DNA molecule or nucleotides, etc. Examples of DNA cleaving reagent include restriction endonucleases.

A “RNA cleaving reagent” is any agent, compound, or substance that can be used to break a bond in a RNA molecule or otherwise restrict a RNA molecule into smaller units, RNA cleavage fragments, e.g., fragments of the RNA molecule or ribonucleotides, etc. Examples of an RNA cleaving reagent include ribozymes and RNAses.

“Probe” refers to a device that, when positionally engaged in interrogatable relationship to a laser desorption ionization source and in concurrent communication at atmospheric or subatmospheric pressure with a gas phase ion spectrometer, can be used to introduce ions derived from an analyte into the spectrometer. As used herein, the “probe” is typically reversibly engageable by a probe interface.

“Affinity capture probe” refers to a probe that binds analyte through an interaction that is sufficient to permit the probe to extract and concentrate the analyte from an inhomogeneous mixture. Concentration to purity is not required. The binding interaction is typically mediated by adsorption of analyte to an adsorption surface of the probe. Affinity capture probes are often colloquially referred to as “protein biochips”, which phrase is thus used herein synonymously with “affinity capture probe”. The term “ProteinChip® array” refers to affinity capture probes that are commercially available from Ciphergen Biosystems, Inc., Fremont, Calif., for use in the present invention. Affinity capture probes can have chromatographic adsorption surfaces or biomolecule affinity surfaces, as hereinafter defined.

“Adsorbent” refers to any material capable of adsorbing an analyte. The term “adsorbent” is used herein to refer both to a single material (“monoplex adsorbent”) (e.g., a compound or a functional group) and to a plurality of different materials (“multiplex adsorbent”). The adsorbent materials in a multiplex adsorbent are referred to as “adsorbent species.” For example, a laser-addressable adsorption surface on a probe substrate can comprise a multiplex adsorbent characterized by many different adsorbent species (e.g., anion exchange materials, metal chelators, or antibodies) having different binding characteristics.

“Adsorption surface” refers to a surface having an adsorbent.

“Chromatographic adsorption surface” refers to a surface having an adsorbent capable of chromatographic discrimination among or separation of analytes. The phrase thus includes surfaces having ion extraction moieties, anion exchange moieties, cation exchange moieties, normal phase moieties, reverse phase moieties, metal affinity capture moieties, and/or mixed-mode adsorbents, as such terms are understood in the chromatographic arts.

“Biomolecule affinity surface” refers to a surface having an adsorbent comprising biomolecules capable of specific binding, such as proteins, oligosaccharides, antibodies, receptors, small molecular ligands, as well as various protein lipo- and glycoconjugates.

The “complexity” of a sample adsorbed to an adsorption surface of an affinity capture probe means the number of different protein species that are adsorbed.

“Specific binding” refers to the ability of two molecular species concurrently present in a heterogeneous (inhomogeneous) sample to bind to one another preferentially over binding to other molecular species in the sample. Typically, a specific binding interaction will discriminate over adventitious binding interactions in the reaction by at least two-fold, more typically more than 10- to 100-fold. When used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

“Gas phase ion spectrometer” refers to an apparatus that measures a parameter which can be translated into mass-to-charge ratios of ions formed when a sample is volatilized and ionized. In the context of this invention, gas phase ion spectrometers include an ionization source used to generate the gas phase ions. Generally ions of interest bear a single charge, and mass-to-charge ratios are often simply referred to as mass. Gas phase ion spectrometers include, for example, mass spectrometers, ion mobility spectrometers, and total ion current measuring devices.

“Gas phase ion spectrometry” refers to a method comprising employing an ionization source to generate gas phase ions from an analyte presented on a sample presenting surface of a probe and detecting the gas phase ions with a gas phase ion spectrometer.

“Mass spectrometer” refers to a gas phase ion spectrometer that measures a parameter which can be translated into mass-to-charge ratios of gas phase ions. Mass spectrometers generally include an inlet system, an ionization source, an ion optic assembly, a mass analyzer, and a detector. Examples of mass spectrometers are time-of-flight, magnetic sector, quadrupole filter, ion trap, ion cyclotron resonance, electrostatic sector analyzer and hybrids of these.

“Mass spectrometry” refers to a method comprising employing an ionization source to generate gas phase ions from an analyte presented on a sample presenting surface of a probe and detecting the gas phase ions with a mass spectrometer.

“Tandem mass spectrometer' refers to any gas phase ion spectrometer that is capable of performing two successive stages of m/z-based discrimination or measurement of ions, including of ions in an ion mixture. The phrase includes spectrometers having two mass analyzers and further includes spectrometers having a single mass analyzer that are capable of selective acquisition or retention of ions prior to mass analysis. The phrase thus explicitly includes QqTOF mass spectrometers, ion trap mass spectrometers, ion trap-TOF mass spectrometers, TOF-TOF mass spectrometers, and Fourier transform ion cyclotron resonance mass spectrometers.

“Laser desorption mass spectrometer” refers to a mass spectrometer which uses a laser as a means to desorb, volatilize, and ionize an analyte. As is well known in the art, laser desorption mass spectrometry of biopolymers typically involves the use of an EAM to facilitate desorption of intact biopolymers for detection.

A “quadrupole time-of-flight mass spectrometer” refers to a mass spectrometer that contains a collisional damping interface that cools the ions formed by the energy source before the ions enter a quadrupole Q. The quadrupole time-of-flight mass spectrophotometer can also contain a collision cell.

“Fluence” refers to the energy delivered per unit area of interrogated image.

“Detect” refers to identifying the presence, absence or amount of the object to be detected.

“Eluant” or “washing solution” refers to an agent that can be used to mediate adsorption of an affinity tagged product to a capture reagent. Eluants and washing solutions are also referred to as “selectivity threshold modifiers.” Eluants and washing solutions can be used to wash and remove unbound materials (e.g., materials not specifically bound) from the probe or substrate surface.

“Adsorption” or “retention” refers to the detectable binding between an absorbent and an affinity tagged product either before or after washing with an eluant (selectivity threshold modifier) or a washing solution.

“Antibody” refers to a polypeptide substantially encoded by at least one immunoglobulin gene or fragments of at least one immunoglobulin gene, that can participate in specific binding with a ligand. The term includes naturally-occurring forms, as well as fragments and derivatives. Fragments within the scope of the term as used herein include those produced by digestion with various peptidases, such as Fab, Fab′ and F(ab)′2 fragments, those produced by chemical dissociation, by chemical cleavage, and recombinantly, so long as the fragment remains capable of specific binding to a target molecule. Typical recombinant fragments, as are produced, e.g., by phage display, include single chain Fab and scFv (“single chain variable region”) fragments. Derivatives within the scope of the term include antibodies (or fragments thereof) that have been modified in sequence, but remain capable of specific binding to a target molecule, including interspecies chimeric and humanized antibodies. As used herein, antibodies can be produced by any known technique, including harvest from cell culture of native B lymphocytes, hybridomas, recombinant expression systems, by phage display, or the like.

Antibodies can be a polyclonal mixture or monoclonal. An “antibody” can be derived from sequence of a mammal, non-mammal (e.g., birds, chickens, fish, etc.), or fully synthetic antibody sequences. A “mammal” is a member of the class Mammalia. Examples of mammals include, without limitation, humans, primates, chimpanzees, rodents, mice, rats, rabbits, sheep, and cows. The term “antibody” also refers to fragments and substitutes for antibodies such as F(ab′)₂, Fab′, and Fab fragments.

Methods of production of polyclonal antibodies are known to those of skill in the art. In brief, an immunogen, preferably a purified protein or biomolecule, is mixed with an adjuvant and animals are immunized. When appropriately high titers of antibody to the immunogen are obtained, blood is collected from the animal and antiserum is prepared.

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host.

“Antigen” refers to a ligand that can be bound by an antibody. An antigen need not be immunogenic. The portions of the antigen that make contact with the antibody are denominated “epitopes”.

An “exogenous gene” is a gene, cDNA, or protein encoding nucleic acid that has been prepared in vitro. An “exogenous gene” can contain sequences with 70% or more identity to nucleic acid sequences from a cell. The “exogenous gene” can be formulated in a plasmid, virus, or expression cassette that contains the necessary elements for expression of protein encoded by the exogenous gene.

A “growth factor” is a compound that stimulates cell division or cell proliferation. Examples of growth factors include, without limitation, EGF, NGF, FGF, etc.

A “chemotherapeutic agent” is a compound or treatment (e.g., x-rays, radiation, etc.) that is used to control the growth of cells, in particular cancers, cancer cells, and tumors. Examples of chemotherapeutic agents include, x-rays, radiation, vincristine, vinblastine, vinorelbine, paclitaxel (Taxol®), methotrexate, daunorubicin, cyclophosphamide, doxorubicin, melphalan, and chlorambucil, cisplatin, Altretamine, Azathioprine, Bleomycin, Busulfan, Carboplatin, CCNU (lomustine), Cladribine, Etoposide, Fluorouracil, Gemcitabine, Hydroxyurea, Idarubicin, Ifosfamide, Interferon, Irinotecan, L-asparaginase, Mercaptopurine, Methyl-CCNU (semustine), Mithramycin, Mitomycin-C, Mitotane, Mitoxantrone Procarbazine, Streptozocin, Tamoxifen, Teniposide, Topotecan, and Trimetrexate.

The term “ultraviolet light” refers to electromagnetic radiation with a wavelength in the range of about 4 to about 400 nanometers.

The term “C₁₋₂₀” refers to a group containing 1 to 20 carbon atoms.

The term “acyl” refers to those groups derived from an organic acid by removal of the hydroxy portion of the acid. Accordingly, acyl is meant to include, for example, acetyl, propionyl, butyryl, decanoyl, pivaloyl, benzoyl and the like. A “C₁-C₂₀ acyl group” is an acyl group having from 1 to 20 carbons.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. a “C₁-C₂₀ alkyl group” is a substituted or unsubstituted alkyl group having from 1 to 20 carbons.). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups, which are limited to hydrocarbon groups are termed “homoalkyl”.

The term “alkylene” by itself or as part of another substituent means a divalent radical derived from an alkane, as exemplified by —CH₂CH₂CH₂CH₂—, and further includes those groups known as “heteroalkylenes.”

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofaran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent, which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and acyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —NR′C(NR′R″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″ and R′″ each independently refer to hydrogen, and heteroalkyl, unsubstituted aryl, aryl substituted with 1-3 halogens, unsubstituted alkyl, alkoxy or thioalkoxy groups, or aryl-(C₁-C₄)alkyl groups. When a compound of the invention includes more than one R group, for example, each of the R groups is independently selected as are each R′, R″ and R′″ groups when more than one of these groups is present. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: —halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NR—C(NR′R″)═NR′″, —NRC(NR′R″)═NR′″, —NR—C(NR′R″)═NR′″, —S(O)R′, —S(O)₂R′, —S(O)₂NR′R″, —NRS(O)₂R′, —N₃, —CH(Ph)₂, fluoro(C₁-C₄)alkoxy, and fluoro(C₁-C₄)alkyl, in number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″ and R′″ groups when more than one of these groups is present.

Two of the substituents on adjacent atoms of the aryl or heteroaryl ring may optionally be replaced with a substituent of the formula —T—C(O)—(CRR′₂)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)ₜ—, where s and t are independently integers of from 0 to 3, and X is —O—, —NR′—, —S—, —S(O)—, —S(O)₂—, or —S(O)₂NR′—. The substituents R, R′, R′ and R′″ are independently selected from hydrogen or unsubstituted (C₁-C₆)alkyl.

As used herein, the term “heteroatom” is meant to include oxygen (O), nitrogen (N), sulfur (S) and silicon (Si).

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention. The chemical compounds of the present invention may exist in (+) and (−) forms as well as in racemic forms.

Racemic forms can be resolved into the optical antipodes by known methods and techniques. One way of separating the racemic forms is exemplified by the separation of racemic amines by conversion of the racemates to diastereomeric salts of an optically active acid. The diastereomeric salts are resolved using one or more art recognized methods. The optically active amine is subsequently liberated by treating the resolved salt with a base. Another method for resolving racemates into the optical antipodes is based upon chromatography on an optical active matrix. Racemic compounds of the present invention can thus be resolved into their optical antipodes, e.g., by fractional crystallization of d- or l-tartrates, -mandelates, or -camphorsulfonate) salts for example.

The chemical compounds of the present invention may also be resolved by the formation of diastereomeric amides by reaction of the chemical compounds of the present invention with an optically active carboxylic acid such as that derived from (+) or (−) phenylalanine, (+) or (−) phenylglycine, (+) or (−) camphanic acid or the like. Alternatively, the compounds of the invention are resolved by the formation of diastereomeric carbamates by reaction of the chemical compound of the present invention with an optically active chloroformate or the like.

Additional methods for the resolving the optical isomers are known in the art. Such methods include those described by Collet and Wilen, ENANTIOMERS, RACEMATES, AND RESOLUTIONS, John Wiley and Sons, New York (1981).

Moreover, some of the chemical compounds of the invention can exist in syn-and anti-forms (Z- and E-form), depending on the arrangement of the substituents around a double bond. A chemical compound of the present invention may thus be the syn- or the anti-form (Z- and E-form), or it may be a mixture hereof.

“Energy absorbing molecules” and the equivalent acronym “EAM” refer to molecules that are capable of absorbing energy from a laser desorption ionization source and thereafter contributing to the desorption and ionization of analyte in contact therewith. The phrase includes molecules used in MALDI, frequently referred to as “matrix”, and explicitly includes cinnamic acid derivatives, sinapinic acid (“SPA”), cyano hydroxy cinnamic acid (“CHCA”) and dihydroxybenzoic acid. It also includes EAM's as described in U.S. Pat. No. 5,719,060 (Hutchens and Yip), the disclosure of which are incorporated herein by reference in its entirety.

### BRIEF DESCRIPTION OF THE DRAWINGS

FIG. 1 depicts three mass spectra displaying the mass range between 2.5 and 15 kDa of affinity tagged products of horseradish peroxidase, bovine IgG, chicken conalbumin, bovine serum albumin and superoxide dismutase. The affinity tagged products were generated by reacting PEO Iodoacetyl Biotin (Pierce Chemical Co.) with proteins in three tubes (Reaction tube A, Reaction tube B, and Reaction tube C). Each tube contained bovine IgG (147.3 kDa; 1.6 nmol), chicken conalbumin (77.49 kDa; 0.6 nmol), bovine serum albumin (66.43 kDa; 0.6 nmol) and superoxide dismutase (15.59 kDa; 0.3 nmol). Horseradish peroxidase was present in different amounts in each tube: 1 nmol in reaction tube A, 2 nmol in reaction tube B, and 3 nmol in reaction tube C. The affinity tagged products were then digested with trypsin and immobilized on streptavidin coated PS2 Protein Chips® (Ciphergen Biosystems, Inc., Fremont, Calif.). The immobilized affinity tagged products were then analyzed using a ProteinChip® System-II (PBSII, Ciphergen Biosystems, Inc.) and the mass spectra are presented in FIG. 1.

FIG. 2 depicts the low mass range, 2.5 kDa to 6 kDa, of the mass spectra of FIG. 1. The arrows indicate peaks corresponding to affinity tagged products of horseradish peroxidase.

FIG. 3A is the capture of biotinylated reporter peptides from Horseradish peroxidase as described in Example 4B. Two reporter fragments were consistently retained on streptavadin coated ProteinChip Arrays. These reporter peptides have masses corresponding to 2955 Da and 3495 Da.

FIG. 3B is the capture of the two biotinylated HRP reporter fragments occurs in a reproducible manner. (A) Seven replicates each of 40 pmol and 61 pmol HRP was spiked into the protein mixture model system. After data collection, (B) and (C) the average and standard deviation was calculated for each of the reporter species at different concentrations. Overall CV was calculated to be 28% for the 2955 Da species and 34% for the 3495 Da species.

FIG. 4 is the capture of the 2955 Da biotinylated reporter peptide of horseradish peroxidase from spiked albumin-depleted human serum.

### DETAILED DESCRIPTION

I. Introduction

The present invention provides for methods and compositions (e.g., capture reagents, affinity tags, substrates, etc.) that are useful for determining the identity and the relative amounts of one or more biomolecules (e.g., proteins, peptides, post-translationally modified proteins, nucleic acids, carbohydrates, lipids, etc.) present in a first and second sample using mass spectrometry. The present invention involves using affinity tags containing an affinity label “A” and a biomolecule reactive group “R.” The affinity tags are used to label one or more proteins by virtue of the biomolecule reactive group R to generate affinity tagged products. The affinity tagged products are then immobilized to a capture reagent on a substrate. The immobilized affinity tagged products on the substrate are then analyzed using mass spectrometry without prior elution or removal of the affinity tagged products from the capture reagent bound to the substrate. The components and methods of the present invention will be described in more detail below.

II. Methods of Present Invention

The methods of the present invention are useful for determining the mass, the amount and identity of biomolecules (e.g., proteins) in a sample. Thus, these methods can be applied in such areas as clinical diagnostics, drug discovery, and drug target discovery. The steps involved in carrying out these will be discussed below, followed by descriptions of various compositions and apparatus that are used to carry out the present invention. These methods typically begin with preparing the samples to be analyzed. These samples may be prepared from a variety of sources and optionally fractionated using biochemical and physical techniques.

After the samples have been prepared they are reacted with an affinity tag that serves to link the affinity tag to a biomolecule through a biomolecule reactive group (i.e., through bonds, including covalent and non-covalent bonds). The affinity tags also contain an affinity label that can specifically bind to a capture reagent. The affinity tags react with biomolecules to form affinity tagged products. These affinity tagged products are then immobilized on a substrate via a capture reagent. The immobilized affinity tagged products can then be treated with a biomolecule cleaving reagent to provide fragments of the affinity tagged products that can be more amenable to mass spectrometry analysis. The immobilized affinity tagged products can also be subjected to wash solutions to remove non-specifically bound material and non-binding material.

Mass spectrometry is then used to analyze the molecules immobilized on the substrate. In preferred embodiments, laser desorption mass spectrometry and tandem mass spectrometry are used to analyze the samples. The mass spectra can then be analyzed to provide information on the relative amount of a biomolecule present in two different samples and in some cases, to provide information on the structure of a biomolecule present in the samples. Thus, these methods are useful in the characterization of biomolecules present in biological samples. The steps involved in the methods of the present invention will now be discussed in further detail.

Sample Preparation

A sample(s) (e.g., a first sample, a second sample, etc.) is in certain some embodiments biological samples. Biological samples are samples derived from a life form such a humans, mice, plants, fungi, yeast, etc. In certain embodiments the samples are derived from humans in the form of a biological sample that comprises one or more biomolecules. Examples of biological samples, include but are not limited to, a blood sample, a urine sample, a cellular lysate, a tumor cell lysate, a saliva sample, a stool sample, a lymphatic fluid sample, a prostatic fluid sample, a seminal fluid sample, a milk sample, and a cell culture medium sample, etc. Typically, the samples are complex biologic samples or moderately complex biologic samples having overlapping biomolecular profiles in which the samples share at least 80% or 90% common biomolecular species. These samples can be processed by lysing, fractionation, purification, etc. before being contacted with an affinity label.

In certain embodiments, the relative amounts of biomolecules in two or more samples are compared. Typically, the samples have overlapping biomolecule profiles. Using the methods of the present invention, the amounts of the biomolecules can be compared to determine how the profiles differ in the nature and amount of biomolecules that are present. These methods are useful for identifying a change in the nature or amount of a biomolecule that is indicative of a disease state (e.g., a disease biomarker, PSA, BRCA1, etc.) or presence of a pathogen (e.g., HIV, bacterial pathogens, viral pathogens, prions, etc), etc. These methods are also useful for discovering biomolecules (e.g., biomakers) that are associated with disease states for drug discovery purposes, diagnostic purposes, etc. In particular, it is useful to compare the biomolecule profiles of samples that are from different subjects or have been subjected to different conditions or treatments. For example, in certain embodiments, the first sample is an untreated control sample and the second sample has been subjected to an agent or condition. Examples of agents include, but are not limited to: a chemotherapeutic agent, ultraviolet light, an exogenous gene, and a growth factor. Those of skill in the art will recognize that there are many ways to introduce an exogenous gene into a cell (see, e.g., Ausubel et al., eds., (1994), supra). In other embodiments, the first sample is a diseased sample and the second sample is a non-diseased sample. In addition, agents can take the form of candidate drugs. For example, the biomolecules in a first sample treated with a candidate drug and can be compared to a second sample which is a negative control. The influence of the candidate drug on the relative amount of a biomolecule (e.g., a protein) present in the first and second sample can be an indication of the candidate drugs efficacy. Those of skill in the art will appreciate that these methods can be adapted to analyze the effects of any agent on a disease state or amount of a disease marker present in a sample. In addition, the identity of molecules in samples can be identified, which can lead to the discovery of new drug targets. In certain embodiments, the methods of the present invention are useful for identifying the presence of a biological agent such as HIV, pathogenic viruses, and pathogenic bacteria in a sample. In addition, the samples analyzed in the present invention can be a cellular lysate. The cellular lysate can be generated from, for example, a prokaryotic cell, a plant cell, a eukaryotic cell, and fungal cell. These cellular lysates can be referred to as a prokaryotic cell lysate, a plant cell lysate, a eukaryotic cell lysate, and a fungal cell lysate, respectively.

Contacting Samples or Affinity Tagged Products with Biomolecule Cleaving Reagents

In certain embodiments, the samples or the affinity tagged products can be contacted with one or more biomolecule cleaving reagents before mass spectrometric analysis. A biomolecule cleaving reagent can be contacted with a sample, an affinity tagged product, or an immobilized affinity tagged product, etc., to cleave the biomolecule of interest. Care should be taken in the choice of and nature of the biomolecule cleaving reagent treatment, so as to not disrupt the immobilization of the affinity tagged product or diminish the activity of the affinity tag. Biomolecule cleaving reagent treatment can have the advantage of providing biomolecule cleavage fragments, which can facilitate mass spectral analysis of the relative amount of biomolecule and the identity of biomolecules in a sample(s). In particular, biomolecule cleaving reagent treatment can facilitate the analysis of biomolecules whose molecular masses exceed 25 kDa.

In a preferred embodiment, polypeptide cleaving reagents are used in the present invention. In a more preferred embodiment, the affinity tagged products are contacted with one or more polypeptide cleaving reagents to generate polypeptide cleavage fragments. A polypeptide cleaving reagent can be contacted with the affinity tagged products before or after the affinity tagged products have been adsorbed to the substrate-bound capture reagent. The polypeptide cleaving reagents can be proteases. Example of proteases that can be used as polypeptide cleaving reagents, include, but are not limited to: chymotrypsin, trypsin, Endoproteinase Glu-C, Endoproteinase Asp-N, Endoproteinase Lys-C, Endoproteinase Arg-C, Endoproteinase Arg-N, Factor Xa protease, thrombin, enterokinase, V5 protease, and the tobacco etch virus protease. Polypeptide cleaving reagents can also include chemical substances and compounds that cleave polypeptides and peptide bonds such as cyanogen bromide (which cleaves at methionine residues), hydroxylamine (which cleaves between an Asn and a Gly residue), and acid pH (which can cleave an Asp-Pro bond) (see e.g., Ausubel et al., supra). The activity of the polypeptide cleaving reagent can be inhibited by treating with heat, protease inhibitor, metal chelator (e.g., EGTA, EDTA), etc.

In other embodiments that involve analyzing samples that contain glycosyl, polysaccharide and carbohydrate residues, a “polysaccharide cleaving reagent” can be used. In certain embodiments, “polysaccharide cleaving reagent” that are glycosidases are used to fragment the biomolecule. Endoglycosidases such as Endoglycosidase H (New England Biolabs, Beverly, Mass.), and Endo H_{f} (New England Biolabs) are commercially available and can be used to restrict glycoproteins, polysaccharides, etc. These endoglycosidases cleave the chitobiose core of high mannose and some hybrid oligosaccharides from N-linked glycoproteins. Exoglycosidases are also commercially available from vendors such as New England Biolabs and include, β-N-Acetylhexosaminidase, α1-2 Fucosidase, α1-3,4 Fucosidase, α1-2,3 Mannosidase, α1-6 Mannosidase, Neuraminidase, α2-3 Neuraminidase, β1-3 Galactosidase, and α-N-Acetyl-galactosaminidase.

In still other embodiments that involve analyzing samples that contain DNA and RNA molecules, a “DNA cleaving reagent” and/or a “RNA cleaving reagent” can be employed to digest the DNA/RNA containing biomolecule. Restriction endonucleases (e.g., Alu I, Ase I, BamH I, Bgl II, Cla I, Dra I, EcoR I, Hind III, Hpa II, Nco I, Not I, Sal I, Sau3A I, Sfi I, Sca I and Sph I) can be used to cleave deoxyribonucleotides that contain the respective restriction endonuclease site.

In still further embodiments, phosphatases (e.g., an alkaline phosphatase, an acid phosphatase, a protein serine phosphatase, a protein tyrosine phosphatase, and a protein threonine phosphatase, etc.), lipases, and other enzymes can be employed as biomolecule cleaving reagents.

Generation of Affinity Tagged Products

The affinity tags described herein are contacted with the samples (e.g., a first sample, a second sample, etc). In certain embodiments, two samples are contacted with the affinity tags in parallel (e.g., separately) and worked up separately (e.g., not combined with each other). The affinity tags can be pre-adsorbed to the capture reagent, or can be free in solution and subsequently immobilized on the capture reagent (see description of immobilization of affinity tagged products below). The biomolecule reactive groups on the affinity tags react with functional groups on biomolecules (e.g., a protein) to form affinity tagged products, which contain bonds between the biomolecules and the affinity tags. In preferred embodiments, the bonds are covalent bonds. In preferred embodiments, the reactions are carried out in an aqueous environment, but can include the presence of organic solvents which may be useful for solubilizing certain affinity tags.

In certain embodiments of the present invention, it may be necessary to pre-treat the samples to be analyzed before being contacted with an affinity tag. Thus, it may be necessary to convert functional groups on the biomolecule into one(s) that are more readily reactive with a particular biomolecule reactive group. For example, vicinal diols on biomolecules (e.g., glycoproteins) can be treated with periodate before subsequent reactions with affinity tags that contain amine or hydrazide containing biomolecule reactive groups. In addition, it may be advantageous to disrupt disulfides and/or denature proteins and other biomolecules in order to permit the biomolecule reactive groups (e.g., a biomolecule reactive group that reacts with sulfhydryls, etc.) to react with the maximum number of potential sites. After the samples have been reacted with affinity tags, the affinity tagged products are then immobilized.

Immobilization of Affinity Tagged Products on Substrates

Immobilization of the affinity tagged products can take place via a capture reagent. The capture reagent is indirectly (e.g., via an adsorbent) or directly immobilized to a substrate. In certain embodiments, samples are immobilized in parallel on positionally distinguishable address (e.g. on different areas on the same object, on different areas on two separate objects, etc.).

In certain embodiments of the invention, affinity tagged products are immobilized by contacting the capture reagent with a substrate that binds to the capture reagent to form a first complex (i.e., a capture reagent/substrate complex), and contacting the first complex with the affinity tagged products to generate a second complex (i.e., an affinity tagged product/capture reagent/substrate complex).

In other embodiments, capture reagent/substrate compositions can be prepared ahead of time for use in sample analysis. Once the capture reagents/substrate compositions have been prepared, they are contacted with an affinity tagged products to form affinity tagged products. The affinity tagged products can contact the substrate-bound capture reagent for period of time sufficient to allow the affinity tagged products to bind to the capture reagent.

In still other embodiments, the affinity tagged products are immobilized by contacting the affinity tagged products with a capture reagent to generate a first complex (i.e., affinity tagged product-capture reagent complexes) and subsequently contacting the first complex with a substrate (or adsorbent coated substrate) that binds to the capture reagent to generate a second complex (i.e., an affinity tagged product/capture reagent/substrate complex). Substrates, can contain substances and coatings (e.g., adsorbents, etc.) that facilitate the binding of a capture reagent.

Typically, the affinity tagged products and the capture reagents are contacted for a period of between about 30 seconds and about 12 hours, and preferably, between about 30 seconds and about 15 minutes.

The temperature at which the affinity tagged products contact the capture reagent can be a function of the particular sample and the selected capture reagent. Typically, the sample is contacted to the probe substrate under ambient temperature and pressure conditions. For some samples, however, temperatures below ambient temperature and/or above the ambient temperature may be optimal. In addition, non-ambient pressure conditions may also prove optimal in a particular assay. The optimal conditions can be determined by one of skill in the art by modulating variables and performing the assay of interest.

Chromatogaphy of Immobilized Affinity Tagged Products

In certain embodiments, the immobilized affinity tagged products on a substrate are treated under conditions that retain the immobilized affinity tagged products and elute materials that are not specifically bound to the capture reagent. The removal of these materials can improve the signal-to-noise ratio of the peaks in the subsequent mass spectrometry.

The conditions that are used to treat the substrates are preferably carried out such that only the immobilized affinity tagged products remain bound on the substrate surface. Washing a substrate surface can be accomplished by, e.g., bathing, soaking, dipping, rinsing, spraying, or washing the substrate surface with an eluant or a washing solution. A microfluidics process is preferably used when a washing solution such as an eluant is introduced to small spots of adsorbents on the probe.

The temperature at which the eluant is contacted to the substrate is a function of the particular affinity tagged products, capture reagent, substrate combination in the embodiment. Typically, the eluant is contacted with immobilized affinity tagged products at a temperature of between 0° C. and 100° C., preferably between 4° C. and 37° C. However, for some eluants, substrates, immobilized affinity products and capture reagent combinations, other temperatures can be optimal and will be readily determinable by those skilled in the art.

Any suitable washing solutions or eluants can be used to wash the substrate surface. For example, organic solutions or aqueous solutions can be used. Preferably, an aqueous solution is used. Exemplary aqueous solutions include a HEPES buffer, a Tris buffer, a phosphate buffered saline (PBS), etc. To increase the wash stringency of the buffers, additives can be incorporated into the buffers. These include, but are limited to, ionic interaction modifiers (e.g., pH, salt type and strength, ionic strength, etc.), non-ionic detergents (e.g., Tween, Trition X-100), surfactants, water structure modifiers (e.g., urea and chaotropic salt solutions, etc.), hydrophobic interaction modifiers, chaotropic reagents, dielectric constant modifiers (e.g., urea, propanol, acetonitrile, ethylene glycol, glycerol, detergents, etc.), affinity interaction displacers, and combinations of the thereof. Specific examples of these additives can be found in, e.g., PCT publication WO 98/59360 and WO 98/59361. The selection of a particular eluant or eluant additives is dependent on other experimental conditions (e.g., capture reagents, adsorbents, substrates, and biomolecules being assayed, etc.) and can be determined by those of skill in the art. The treatment of the substrates should be carried out to minimize the loss of immobilized affinity target products and maximize the loss of non-specifically bound material.

III. Mass Spectrometry Methods

The immobilized affinity tagged products on the substrate, washed or unwashed, are then subjected to mass spectrometry after being contacted with an energy absorbing molecule. One advantage of the present invention is that the immobilized affinity tagged products are retained on the substrate and are not eluted for subsequent mass spectrometric analysis. Thus, unlike methods that employ conventional chromatography, which requires elution of the desired analyte prior to detection, immobilized affinity tagged products do not need to be transferred to new format. In addition, there is no routine or convenient means for detecting an immobilized affinity tagged product which is not eluted off an adsorbent as in conventional chromatography. Thus, the methods of the present invention provide direct information about chemical or structural characteristics of the retained immobilized affinity tagged products.

The affinity tagged polypeptides of the invention or fragments thereof are analyzed using mass spectrometry methods. Mass spectrometry methods have been used to quantify and/or identify biomolecules, such as proteins (see, e.g., Li et al. (2000) Tibtech 18:151-160; Rowley et al. (2000) Methods 20: 383-397; and Küster and Mann (1998) Curr. Opin. Structural Biol. 8: 393-400). Mass spectrometric techniques have also been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample on the immobilized affinity tagged products. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte, which may contain biological molecules, is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the affinity tagged products. In another variant, the surface is derivatized with energy absorbing molecules that are not desorbed when struck with the laser. In another variant, the surface is derivatized with molecules that bind the affinity tagged products and contain a photolytic bond that is broken upon application of the laser. In each of these methods, the derivatizing agent generally is localized to a specific location on the substrate surface where the sample is applied. See, e.g., U.S. Pat. No. 5,719,060 (Hutchens & Yip) and WO 98/59361 (Hutchens & Yip). The two methods can be combined by, for example, using a SELDI affinity surface to capture an analyte and adding matrix-containing liquid to the captured analyte to provide the energy absorbing material.

In one embodiment, a mass spectrometer is used with a substrate of the present invention. A sample placed on a feature of the substrate of the present invention is introduced into an inlet system of the mass spectrometer. The sample is then ionized by an ionization source. Typical ionization sources include, e.g., laser, fast atom bombardment, or plasma. The generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of an analyte will typically involve detection of signal intensity. This, in turn, reflects the quantity of analyte bound to the substrate. For additional information regarding mass spectrometers, see, e.g., Principles of Instrumental Analysis, 3^{rd} ed., Skoog, Saunders College Publishing, Philadelphia, 1985; and Kirk-Othmer Encyclopedia of Chemical Technology, 4^{th} ed. Vol. 15 (John Wiley & Sons, New York 1995), pp. 1071-1094.

In one embodiment, a mass spectrometer is used to detect affinity tagged products on the substrate. In a typical mass spectrometer, a substrate containing affinity tagged products is introduced into an inlet system of the mass spectrometer. The analyte is then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector.

The detector then translates information of the detected ions into mass-to-charge ratios. Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis (see below), etc.), to determine the relative amounts of particular biomolecules. The mass spectrometers and their techniques are well known to those of skill in the art. Any person skilled in the art understands, any of the components of a mass spectrometer (e.g., desorption source, mass analyzer, detect, etc.) can be combined with other suitable components described herein or those known in the art.

In a preferred embodiment, a laser desorption time-of-flight mass spectrometer is used with the substrate of the present invention. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In certain embodiments, mass spectrometry is used to detect complex formation between the affinity tagged products and the capture reagent bound to the substrate. The complex formation can be optimized by monitoring the amount of signal strength of a particular peak of a mass-to-charge ration in relation to the experimental conditions being used (e.g., the adsorbent, the substrate, wash conditions, the affinity tagged products being analyzed, etc.). The masses of the affinity tagged products in the sample can readily be determined to arrive at an accurate molecular mass of the affinity tagged products in the sample. If one of the masses is an affinity tagged product (e.g., an affinity tagged protein), then the molecular mass of the affinity tag can be subtracted from the parent molecular mass to arrive at the mass of the product (e.g., the protein) itself. The identity (i.e., its structure) of the product (e.g., protein) can be determined using bioinformatic techniques (see below).

Additionally, the polypeptide analytes bound to the substrate can be fragmented with a biomolecule cleaving reagent (e.g., polypeptide cleaving reagent) so that the molecular masses of the biomolecule cleaving reagent fragments can be determined. These molecular masses can be compared to those in a database to provide additional confidence that the proper biomolecule has been identified (see Data Analysis section, below).

Tandem Mass Spectrometry

Tandem mass spectrometry (e.g., MS/MS, MS/MS/MS, ESI-MS/MS, etc.) can also be employed to obtain sequence information on biomolecules such as proteins and peptides. Tandem mass spectrometry refers to a group of mass spectrometry methods that generate a parent ion, which is subsequently fragmented into daughter ion(s) which are then mass analyzed. Typically, a mass filter is used to select parent ions with a particular mass-t0-charge ration for fragmentation. In certain embodiments, the fragmentation is collision-induced dissociation (CID). CID can be carried out in a collision chamber located between a first mass spectrometer and a second mass spectrometer. The collision chamber is filled with a buffer gas, typically an inert gas such as helium. Alternatively, a parent ion can be fragmented using surface induced dissociation, photodissociation (e.g., with lasers), and electron induced dissociation (e.g., with electron beams).

In mass spectrometers that employ time-of-flight analyzers, post-source decay (PSD) and in-source decay (ISD) have been used to generate fragmentation events (see e.g., Li et al. (2000) supra). PSD involves “filtering” a precursor ion from a peptide ion composition using a timed-ion-selector. The selected mass ion spontaneously decays into fragment ions that are separated in the reflectron. ISD uses different conditions that PSD and involves a fast metastable decay in the ion source.

In preferred embodiments, tandem mass spectrometry is carried out using a laser desorption/ionization mass spectrophotometer that is further coupled to a quadrupole time-of-flight mass spectrometer QqTOF MS (see e.g., Krutchinsky et al., WO 99/38185). Methods such as MALDI-QqTOFMS (Krutchinsky et al., WO 99/38185; Shevchenko et al. (2000) Anal. Chem. 72: 2132-2141), ESI-QqTOF MS (Figeys et al. (1998) Rapid Comm'ns. Mass Spec. 12-1435-144) and chip capillary electrophoresis (chip-CE)-QqTOF MS (Li et al. (2000) Anal. Chem. 72: 599-609) have been described previously.

IV. Data Analysis of Mass Spectra

The mass spectra data obtained using the mass spectrometry methods of the present invention can be used to obtain information on the quantity and identity of the affinity tagged products. Data generated by desorption and detection of polypeptides can be analyzed using any suitable means (e.g., visually, by computer, etc). In one embodiment, data is analyzed with the use of a programmable digital computer. The computer program generally contains a readable medium that stores codes. Certain code can be devoted to memory that includes the location of each feature on a substrate, the identity of the adsorbent at that feature and the elution conditions used to wash the adsorbent. Using this information, the program can then identify the set of features on the substrate defining certain selectivity characteristics (e.g., types of adsorbent and eluants used). The computer also contains code that receives as input, data on the strength of the signal at various molecular masses received from a particular addressable location on the substrate. This data can indicate the number of affinity tagged products detected, optionally including the strength of the signal of a peak value and the determined molecular mass for each affinity tagged product detected.

Data analysis can include the steps of determining signal strength (e.g., height of peaks) of a peak value (e.g., of a particular mass-to-charge value or range of values) detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each polypeptide or other substances can be displayed in the form of relative intensities in the scale desired (e.g., 100). Alternatively, a standard may be admitted with the sample so that a peak from the standard can be used as a reference to calculate relative intensities of the signals observed for each affinity tagged product detected. Software programs such as the Biomarker Wizard program (Ciphergen Biosystems, Inc., Fremont, Calif.) can be used to aid in analyzing mass spectra.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined (see below).

The present invention also provides methods of determining the identity of a biomolecule (e.g., protein). In certain embodiments, a programmable digital computer is used to access a database containing one or more mass spectra. An algorithm is then executed with a programmable digital computer to determine at least a first measure for each of the predicted mass spectra. The first measure is an indication of the closeness-of-fit between a mass spectrum of the biomolecule and each of the plurality of predicted mass spectra.

The data of a mass spectrum can be used to identify the proteins and biomolecules present in a sample by executing an algorithm with a programmable digital computer that compares the MS data to records in a database. Each molecule provides characteristic mass-spectrometric (MS) data (also referred to as a mass spectral “signature” or “fingerprint”) when analyzed by MS methods. This data can be analyzed by comparing it to databases containing, inter alia, actual or theoretical MS data or biomolecule sequence information. Additionally, a biomolecule may be cleaved into fragments for MS analysis. Information obtained from the MS analysis of fragments is also compared to a database to identify biomolecules (e.g., proteins) in the sample (see e.g., Yates (1998) J. Mass Spec. 33:1-19; Yates et al., U.S. Pat. No. 5,538,897; Yates et al., U.S. Pat. No. 6,017,693; WO 00/11208 and Gygi et al. (1999) Nat. Biotechnol. 10:994-999). Software resources that facilitate interpretation of mass spectra, especially protein mass spectra, and mining of public domain sequence databases are now readily accessible on the Internet to facilitate protein identification. Among these are Protein Prospector (http://prospector.ucsf/edu), PROWL (http://prowl.rockefeller.edu), and the Mascot Search Engine (Matrix Science Ltd., London, UK, www.matrixscience.com).

In certain embodiments, MS data and information obtained from that data are compared to a database consisting of data and information relating to biomolecules. For example, the database may consist of sequences of nucleotides or amino acids. The database may consist of nucleotide or amino acid sequences of expressed sequence tags (ESTs). Alternatively, the database may consist of sequences of genes at the nucleotide or amino acid level. The database can include, without limitation, a collection of nucleotide sequences, amino acid sequences, or translations of nucleotide sequences included in the genome of any species.

A database of information relating to biomolecules, e.g., sequences of nucleotides or amino acids, is typically analyzed via a computer program or a search algorithm which is optionally performed by a computer. Information from sequence databases is searched for best matches with data and information obtained from the methods of the present invention (see e.g., Yates (1998) J. Mass Spec. 33: 1-19; Yates et al., U.S. Pat. No. 5,538,897; Yates et al., U.S. Pat. No. 6,017,693).

Any appropriate algorithm or computer program useful for searching a database can be used. Search algorithms and databases are constantly updated, and such updated versions will be used in accordance with the present invention. Examples of programs or databases can be found on the World Wide Web (WWW) at http://base-peak.wiley.com/, http://mac-mann6.embl-heidelberg.de/MassSpec/Software.html, http://www.mann.embl-heidelberg.de/Services/PeptideSearch/PeptideSearchIntro.html, ftp://ftp.ebi.ac.uk/pub/databases/, and http://donatello.ucsf.edu. U.S. Pat. Nos. 5,632,041; 5,964,860; 5,706,498; and 5,701,256 also describe algorithms or methods for sequence comparison.

In one embodiment, the database of protein, peptide, or nucleotide sequences is a combination of databases. Examples of databases include, but are not limited to, ProteinProspector at the UCSF web site (prospector.ucsf.edu), the Genpept database, the GenBank database (described in Burks et al. (1990) Methods in Enzymology 183: 3-22, EMBL data library (described in Kahn et al. (1990) Methods in Enzymology 183:23-31, the Protein Sequence Database (described in Barker et al. (1990) Methods in Enzymology 183: 31-49, SWISS-PROT (described in Bairoch et al. (1993) Nucleic Acids Res., 21: 3093-3096, and PIR-International (described in (1993) Protein Seg. Data Anal. 5:67-192).

In a further embodiment, novel databases are generated for comparison to mass spectrometrically determined MS data, e.g., mass or mass spectra of cleaved protein and peptide fragments. For example, a theoretical database of all the possible amino acid sequence combinations of the peptide masses being characterized is generated (Parekh et al., WO 98/53323). Then, the database is compared with the actual masses determined using mass spectrometry to determine the amino acid sequence of the peptides in the sample.

In some embodiments, the mass of a polypeptide derived from a mass spectrum is used to query a database for those masses of proteins or predicted proteins from nucleic acid sequences that provide the closest fit. In this manner, an unknown protein can be rapidly identified without an amino acid sequence. In other embodiments of the invention, the masses provided from polypeptide fragments thereof can be compared to the predicted mass spectra of a database of proteins or predicted proteins from a nucleic acid sequences that provide the closest fit. An algorithm or computer program generates a theoretical cleavage of sequences in a database with the same cleavage agent used to cleave the biomolecule analyzed by MS methods. In addition, the nature of the biomolecule reactive group being used can aid in identifying the biomolecule in the affinity tagged product. For example, if the biomolecule reactive group is specific for a particular functional group then biomolecules that lack that particular functional group can probably be eliminated as possibilities for the affinity tagged product. For example, if biomolecule reactive group reactive with sulfhydryls is used, then proteins that lack cysteine can probably be eliminated as candidates for the affinity tagged product.

Sequences or simulated cleavage fragments from the sequence database that fall within a desired range of similar sequence homologies to sequences generated from the MS data of parent or fragment molecules are designated “matches” or “hits.” In this manner, the identity of the biomolecules or fragments thereof can be rapidly determined. The investigator can customize or vary the range of acceptable sequence homology comparison values according to each particular analysis.

V. Systems

The present invention also encompasses systems for characterizing a biomolecules. In certain embodiments, the system contains a gas phase ion spectrophotometer, a substrate (e.g., a chip containing an adsorbent), a capture reagent, an affinity tag and a computer with a local database capable of storing at least one mass spectrum record. The systems can also contain an input device, an output device, and a computing device electrically coupled to the input device. The output device and the local database are configured to receive mass spectra data from the mass spectrophotometer. The computing device is used to execute an algorithm which compares the sequence records in the database or compares a first mass spectrum of a first sample to a second mass spectra of a second sample. In other embodiments, a system can contain a remote computing device configured to receive mass spectra data from the mass spectrophotometer via a computer network. The remote computing device is coupled to a remote database containing mass spectra or biomolecule sequence records, a remote output device and a remote input device. The remote computing device contains code for executing an algorithm which compares mass spectrum record(s) or biomolecule sequence records in the database to the mass spectra of the sample.

VI. Affinity Tags

In the present invention, the affinity tags contain an affinity label (“A”) attached to a biomolecule reactive group (“R”) to form compounds with the structure of A—R. In some embodiments, the affinity tags also contain a linker (L) to form compounds having the formula A—L—R. The affinity tags need not be labeled with isotopes (e.g., ¹³C, ²H, ³⁴S, ¹⁸O, ¹⁷O, ¹⁵N, etc.).

Affinity Labels

The affinity labels are able to specifically bind to a capture reagent. Thus, affinity tagged products by virtue of the affinity label portion of the affinity tag can be bound to a capture reagent. Essentially any molecule that can be attached to a biomolecule reactive group and can selectively bind to a capture reagent can be employed as an affinity label. Examples of affinity labels include, but are not limited to, biotin, iminobiotin, glutathione, maltose, a nitrilotriacetic acid group, a polyhistidine group, an oligonucleotide, and a hapten (see also, WO 00/11208 for descriptions of affinity labels). A list of examples of capture reagent-affinity label pairs are set out in the Table 1:

TABLE 1

| Affinity label | Capture Reagent |
|---|---|
| biotin | avidin, streptavidin, NeutrAvidin biotin binding protein |
| | (Molecular Probes), Streptavidin agarose (Molecular Probes), |
| | CaptAvidin agarose (Molecular Probes), CaptAvidin biotin |
| | binding protein (Molecular Probes), Streptavidin acrylamide |
| | (Molecular Probes), or an anti-biotin antibody |
| iminobiotin | avidin, streptavidin, NeutrAvidin biotin binding protein |
| | (Molecular Probes), Streptavidin agarose (Molecular Probes), |
| | CaptAvidin agarose (Molecular Probes), CaptAvidin biotin |
| | binding protein (Molecular Probes), Streptavidin acrylamide |
| | (Molecular Probes), or an anti-biotin antibody |
| glutathione | glutathione S-transferase |
| maltose | maltose binding protein |
| nitrilotriacetic acid group | polyhistidine |
| a polyhistidine group | nitrilotriacetic acid group |
| an oligonucleotide | the complementary oligonucleotide |
| hapten | an anti-hapten antibody |
| digoxigenin | an anti-digoxigenin antibody |
| dinitrophenyl group | an anti-dinitrophenyl antibody |
| fluorescein | an anti-fluorescein antibody |

In certain embodiments, biotin and iminobiotin are preferred due to the availability large number of capture reagents that are known to bind biotin and are commercially available (see, e.g., Table 1). In addition, affinity tags that contain biotin or iminobiotin as affinity labels are commercially available from vendors (e.g., Pierce Chemical Co., Molecular Probes, etc.)

Oligonucleotides can be used as affinity label and capture reagent pairs. Oligonucleotides include nucleic acids such as DNA, RNA, and mixed RNA/DNA molecules. The oligonucleotide that is used as the affinity label should be able to hybridize to the sequence of the oligonucleotide present on the capture reagent. Those of skill in the art will recognize that many different oligonucleotide sequences can be designed that will hybridize to each other and can serve as capture reagent/affinity label pairs. Important considerations for designing such oligonucleotide pairs include the actual nucleotide sequence, the length of the oligonucleotides, the hybridization conditions (e.g., temperature, salt concentration, presence of organic chemicals, etc.) and the melting temperature of the oligonucleotide pairs. It is preferred that the length of the oligonucleotides be at least 7 base pairs, more preferably at least 10 base pairs, still more preferably at least 15 base pairs. The oligonucleotide pairs should possess at least 50% identity, more preferably, 70% identity, and still more preferably 100% identity. The terms “identical” or percent “identity,” in the context of two or more nucleic acids sequences, refer to two or more sequences or subsequences that are the same or have a specified percentage of nucleotides (i.e., 70% identity) that are the same, when compared and aligned for maximum correspondence over a comparison window, as measured using one of the following sequence comparison algorithms or by manual alignment and visual inspection. Such sequences are then said to be “substantially identical.”

In other embodiments of the present invention, affinity labels that contain a nitrilotriacetic acid (NTA) group are used. Typically, capture reagents that contain a polybistidine (e.g., 6 or more histidines, preferably 10 histidines, more preferably 12 histidines) molecule are used to bind affinity labels containing a nitriloacetic acid group. The presence of a metal (e.g., Nickel, etc.) is necessary for the binding of a NTA group to polyhistidine. (see Current Protocols in Molecular Biology (Ausubel et al., eds., (1994)) for a review of immobilized metal affinity technology). Capture reagents containing Ni-NTA groups (nickel-nitrilotriacetic acid) such as Ni-NTA Agarose (Qiagen) or Ni-NTA Magnetic Agarose Beads (Qiagen) can be used to bind affinity tags and affinity tagged products that have a polyhistidine molecule. Those of skill in the art will recognize that polyhistidine can be used as an affinity label and NTA and Ni-NTA containing molecules can be used as capture reagents.

In other embodiments of the present invention, antigens are used as affinity labels and antibodies are used as a capture reagent that will specifically bind to the antigen. An “antigen” is a molecule that is recognized and bound by an antibody, e.g., peptides, carbohydrates, organic molecules, or more complex molecules such as glycolipids and glycoproteins. The part of the antigen that is the target of antibody binding is an antigenic determinant and a small functional group that corresponds to a single antigenic determinant is called a hapten. A hapten is a compound, typically of low molecular weight (e.g., less than 10,000 kDa) that is not immunogenic when injected into an animal, but can induce an immunogenic response and anti-hapten antibody formation when bound to a carrier protein (e.g., Keyhole Limpet Hemocyanin, etc.) or cell, becomes immunogenic and induces antibodies, which can specifically bind the hapten or a hapten-carrier conjugate. There are many examples of haptens that are known in the art (e.g., a dinitrophenyl group, digoxigenin, fluorophores, Oregon Green dyes, Alexa Fluor 488 (Molecular Probes), fluorescein, a dansyl group, Marina Blue (Molecular Probes), tetramethylrhodamine, Texas Red (Molecular Probes), BODIPY (4,4-difluoro-4-bora-3a,4a-diaza-s-indacene; U.S. Pat. No. 4,774,339) dyes, etc) that can be used as affinity labels in the present invention. Antibodies that can be used as capture reagents can specifically bind to haptens are commercially available from vendors such as Molecular Probes, Eugene, Oreg. These antibodies include antibodies that can specifically bind to a dinitrophenyl group, a digoxigenin, a fluorophore, Oregon Green dyes, Alexa Fluor 488 (Molecular Probes), fluorescein, a dansyl group, Marina Blue (Molecular Probes), tetrahmethylrhodamine, Texas Red (Molecular Probes), and a BODIPY dye (Molecular Probes). In some embodiments, the presence of a chromophoric hapten on an affinity tag or on an affinity tagged product can be detected using spectrophotometric or fluorometric means.

Biomolecule Reactive Groups

The contacting of biomolecules with molecules containing biomolecule reactive groups in an aqueous or mixed aqueous/organic solvent milieu is a technique known in the art. See, for example, Means et al., CHEMICAL MODIFICATION OF PROTEINS, Holden-Day, San Francisco, 1971; Feeney et al., MODIFICATION OF PROTEINS: FOOD, NUTRITIONAL AND PHARMACOLOGICAL ASPECTS, Advances in Chemistry Series, Vol. 198, American Chemical Society, Washington, D.C., 1982; Feeney et al., FOOD PROTEINS: IMPROVEMENT THROUGH CHEMICAL AND ENZYMATIC MODIFICATION, Advances in Chemistry Series, Vol. 160, American Chemical Society, Washington, D.C., 1977; Hermanson, BIOCONJUGATE TECHNIQUES, Academic Press, San Diego, 1996 and WO 00/11208.

The biomolecule reactive groups on the affinity tags typically are selected from the variety of well-known moieties, groups and reagents that have the ability to form bonds (e.g., covalent bonds, etc.) by reacting with functional groups on proteins, derivatized proteins and biomolecules (e.g., periodate treated proteins, etc.), and other biomolecules. Examples of functional groups, include without limitation, primary amines, secondary amines, hydroxyls, amines, imidazole rings, carboxylates, sulfhydryls, disulfides, thioethers, imidazolyls, phenol rings, indolyl rings, guanidinyl groups, vicinal diols, aldehydes, etc.

Sulfhydryl Biomolecule Reactive Groups

Sulfhydryl groups (e.g., on cysteine side chains) can be reacted with biomolecule reactive groups such as activated acyl groups, activated alkyl groups, pyridyl-disulfide groups, malemide groups, etc. (see e.g., Hermanson, supra). Examples of sulfhydryl biomolecule reactive groups include iodoacetamide groups, maleimide groups, alkyl halides, aryl halides, sulfonyl halides, nitrites, α-haloacyl groups, epoxides, etc. (see e.g., Hermanson, supra; Feeney et al., MODIFICATION OF PROTEINS: FOOD, NUTRITIONAL AND PHARMACOLOGICAL ASPECTS, supra).

Cystine molecules can be reduced to form sulfhydryl containing cysteines using disulfide disrupting agents, such as reducing agents, β-mercaptoethanol, 2-mercaptoethylamine.HCl (MEA; Pierce Chemical Co., Product No. 20408), dithiothreitol (DTT; Pierce Chemical Co., Product No. 20290), dithioerythritol (DTE), glutathione, thiogylcolate, tris-(2-carboxyethyl)phosphine, and tributyl phosphine (5 mM; Fluka, 90827). In certain embodiments, it may be necessary to use a denaturing agent, such as heat, guanidine HCl (Sigma, G7153), urea, etc., in conjunction with a disulfide disrupting agent to maximize the number of accessible sulfhydryls that are subsequently reacted with a sulfhydryl biomolecule reactive group. In addition, it is necessary to remove many disulfide disrupting agents, by dialysis, washing, etc. Disulfide disrupting agents can interfere with subsequent reactions that involve sulfhydryl biomolecule reactive groups.

In other embodiments, disulfide bonds can be disrupted to form sulfhydryls using immobilized disulfide reactants (Hermanson, supra). These reactants can contain a solid support modified with compounds that contain sulfhydryl groups such as N-acetyl-homocysteine, cysteine, or dihydrolipolipoamide, attached to a spacer molecule such as a diaminopropylamine spacer.

Hydroxyl Biomolecule Reactive Groups

Hydroxyl biomolecule reactive groups can be used to react with hydroxyls that occur on biomolecules, such as the hydroxyl on a tyrosyl side chain. Hydroxyls on tyrosyl side chains can be reacted biomolecule reactive groups such as active alkylating reagents, and active acylating agents (see e.g., Hermanson, supra). In addition, epoxides and oxiranes can be used as hydroxyl biomolecule reactive groups to couple affinity labels to with hydroxyls to create ether bonds. Alternatively, hydroxyl groups can be converted into functional groups that can react with other biomolecule reactive groups. For example, carbonyldiimidazoles can be reacted with hydroxyls to create imidazole carbamates that will react with an amine biomolecule reactive group to create a carbamate linkage. Also, N,N′-disuccinimidyl carbonate can be used to convert hydroxyls on biomolecules to a succinimidyl carbamate (see e.g., Hermanson, supra). The succinimidyl carbamate is then reactive towards amine containing biomolecule reactive groups.

Imidazolyl and Phenol Ring Biomolecule Reactive Groups

Imidazolyl and phenol ring biomolecule reactive groups can be used to react the affinity labels with imidazolyl and phenol rings such as those on histidine and tyrosine side chains. Diazotization of aromatic amino groups, such as those on the affinity label Nα-(4-aminobenzyoylbiocytin)(Molecular Probes, Eugene, Oreg.), results in the formation of imidazolyl, phenol ring, and guanosine reactive groups (see e.g., Hermanson, supra); p-aminobenzoyl biocytin is treated with sodium nitrite to form a diazonium group, which in turn reacts with residues such as histidine and tyrosine to form covalent diazo bonds.

Carboxylate Biomolecule Reactive Groups

Biomolecule reactive groups that specifically react with carboxylate functional groups are known in the art (see, e.g., Hermanson, supra). Carboxylate biomolecule reactive groups include, but are not limited to, diazoalkanes, diacetyl compounds, and carbonyldiimidazoles. Carbodiimides can be used to activate carboxylate groups for subsequent reaction with amine biomolecule reactive groups.

Amine Biomolecule Reactive Groups

In certain embodiments, amine biomolecule reactive groups, including but not limited to, succinimidyl esters, N-hydroxysuccinimidyl (NHS) esters, sulfosuccinimyl esters, isothiocyanates, isocyanates, sulfonyl chlorides, dichlorotriazines, aryl halides, and acyl azides, etc. Primary amine reactive groups include activated acyl groups and activated alkyl groups (see, e.g., Hermanson, supra). In addition, pentafluorophenyl esters and tetrafluorophenyl esters (e.g., 4-sulfo-2,3,5,6-tetrafluorophenyl ester, etc.) can react with primary and secondary amines to form covalent bonds.

Derivatization of Proteins to Create Biomolecule Reactive Groups

Functional groups on biomolecules such as vicinal diols on glycoproteins, polysaccharides, RNA, N-terminal serine resides, N-terminal threonine residues, etc., can be reacted with compounds that will derivatize or convert the functional group into a different functional group that can react with a particular biomolecule reactive group on an affinity label. For example, vicinal diols on biomolecules can be treated with periodate to create aldehyde residues (see, e.g., Hermanson, supra). These aldehyde residues can be reacted with affinity tags that contain biomolecule reactive groups with amine or hydrazide moieties (e.g., 6-((6-((biotinoyl)amino)hexanoyl) amino)hexanoic acid, hydrazide (biotin-XX hydrazide; Cat. No. B-2600; Molecular Probes, Inc.), and biocytin hydrazide (L-Lysine, N6-[5-(hexahydro-2-oxo-1H-thieno[3,4-d]imidazol-4-yl)-1-oxopentyl]-, hydrazide, [3aS-(3aα,4β,6aα)]-; Cat. No. B-1603; Molecular Probes, Inc.), etc.). In addition, a basic sites on nucleotides (e.g., apurinic and apyrimidinic lesions) contain aldehydes that can be reacted with the hydroxlamine biomolecule reactive group on the membrane permeant affinity label ARP (N-(aminooxyacetyl)-N′-(D-biotinoyl)hydrazine, trifluoroacetic acid salt; Cat. No. A-10550; Molecular Probes). Also, biomolecules that contain galactose residues can be treated with galactose oxidase to form aldehydes. This method has been used to oxidize galactose residues on glycoproteins for subsequent reactions with hydrazine and amine containing biomolecule reactive groups.

Biomolecule Reactive Groups that Target Biomolecule Classes

Biomolecule reactive groups can also target classes of biomolecules with a particular ligand binding or enzymatic property. For example, affinity labeling of purine nucleotide binding proteins is known in the art (see Colman, (1983) Ann. Rev. Biochem. 52: 67-91). In particular the biomolecule reactive group 5′-FSBA(5′-(4-Fluorosulfonylbenzoyl)adenosine (Aldrich), has found wide applicability in affinity labeling of ATP-binding molecules such as protein kinases (cAMP-dependent protein kinase, cGMP-dependent protein kinase, casein kinase, EGF receptor kinase, etc.), gluatamate dehydrogenase, 3α, 20β-hydroxysteroid dehydrogenase, gluatamine synthetase, etc. (Colman, supra). Compounds structurally related to 5′ FSBA (e.g., 3′-FSBA, 5-p-fluorosulfonylbenzoyl guanosine, etc.) have also proved useful as biomolecule reactive groups for labeling of nucleotide binding proteins (Colman, supra). Thus, affinity labels of the present invention that contain FSBA and FSBA-like biomolecule reactive groups can be used to characterize FSBA and FSBA-like reactive biomolecules in a sample. Such affinity labels can be useful for analyzing the activation of kinase cascades such as those affected by tumor suppressors, growth factors, etc.

Linkers

A and R can be connected to each other either directly or, alternatively, they can be connected through a linker L to form compounds having the formula A—L—R. Linkers of use in the present invention can have a range of structures, substituents and substitution patterns (see e.g., WO 00/11208). They can, for example be derivatized with nitrogen, oxygen and/or sulfur containing groups which are pendent from, or integral to, the linker group backbone. Examples of linkers include, without limitation, an amide, a polyethylene oxide, a polyethylene glycol, a polyether, a polyether diamine, a diamine, a polyamide, a polythioether, a silyl ether, an alkyl, an alkylenyl, and an alkyl-aryl group. The linkers can contain one or more heteroatoms (e.g., N, O, or S atoms). Examples of linkers useful in the present invention include those present in the affinity tags described and referenced herein (e.g., [+]-biotinyl-iodoacetamidyl-3,6-dioxaoctanediamine, etc.). In certain embodiments, the linkers are comprised of C₁₋₂₀ molecules. In certain embodiments, a linker contains a C₁₋₂₀ amide, a C₁₋₂₀ polyethylene oxide, a C₁₋₂₀ polyethylene glycol, a C₁₋₂₀ polyether, a C₁₋₂₀ polyether diamine, a C₁₋₂₀ diamine, a C₁₋₂₀ polyamide, a C₁₋₂₀ polythioether, a C₁₋₂₀ silyl ether, a C₁₋₂₀ alkyl, a C₁₋₂₀ alkylenyl, or a C₁₋₂₀ alkyl-aryl group. Linkers can serve the purpose of facilitating the reaction of the biomolecule reactive group to a functional group and/or the binding of the capture reagent to the affinity label.

Procuring Affinity Tags

Many affinity tags can be purchased through commercial vendors such as Molecular Probes of Eugene, Oreg., and Pierce Chemical Co. of Rockford, Ill. (see description of some of these tags below). In addition, the affinity tags that are useful in the present invention can be synthesized. Many synthetic routes for making A—R affinity tags and A—L—R affinity tags are known to those of skill in the art and one of skill would be able to choose appropriate reaction conditions for the formation of these kinds of tags. See, for example, Sandler et al ORGANIC FUNCTIONAL GROUP PREPARATIONS 2^{nd} Ed., Academic Press, Inc. San Diego 1983; WO 00/11208. In addition, the synthesis of sulfo-N-hydroxy succinimide affinity tags are described in the art (see, e.g., Hermanson, supra; WO 00/11208; and U.S. Pat. Nos. 5,942,628, 5,892,057, and 5,872,261). WO 00/11208 describes the synthesis of affinity tags (e.g., biotinyl-iodoacetylamidyl-4,7,10 trioxatridecanediamine, etc.) that can target functional groups on biomolecules including sulfhydryl and amine functional groups. U.S. Pat. No. 5,872,261 describes methods for the preparation of sulfo-N-hydroxysuccinimides that involves esterifying a sulfo-succinic acid, which is then reacted with a hydroxylamine to form a sulfo-N-hydroxy succinimide. The affinity tags that are useful in the present invention can be composed of combinations of the affinity labels, linkers and biomolecule reactive groups described herein. Ideally, such combinations should result in an affinity tag that can react with a biomolecule to produce affinity tagged products which can be specifically bound by a capture reagent and subsequently analyzed using mass spectrometry. In certain preferred embodiments, the affinity tags contain biotin and are commercially available. These biotin containing affinity tags are available from vendors such as Molecular Probes and Pierce Chemical Co., and can target a variety of functional groups on biomolecules. The following is a brief synopsis of these kinds of affinity tags.

Biotin Containing Affinity Tags for Amines

EZ-Link™ PFP-Biotin (Pierce Chemical Co.) contains pentafluorophenyl esters that can react with primary and secondary amines to form affinity tagged biomolecules, proteins, peptides, etc. Similarly, EZ-Link™ PEO-Biotin (Pierce Chemical Co.) is reactive towards primary and secondary amines by virtue of its tetrafluorophenyl ester group. EZ-Link™ PEO-Biotin also has good solubility in aqueous solution due to the polyethylene oxide linker group that connects the tetrafluorophenyl group to the biotin group.

Molecular Probes, Inc. is another amine reactive biotinylation vendor and provides such reagents as: B-1513, succinimidyl D-biotin, 2,5-Pyrrolidinedione, 1-((5-hexahydro-2-oxo-1H-thieno(3,4-d)imidazol-4-yl)-1-oxopentyl)oxy)-(3aS-(3a-α,4β,6a-α))-; Cat. No. B-1582, 6-((biotinoyl)amino)hexanoic acid, succinimidyl ester, 1H-Thieno(3,4-d)imidazole-4-pentanamide, N-(6-((2,5-dioxo-1-pyrrolidinyl)oxy)-6-oxohexyl)hexahydro-2-oxo, (3aS-(3a-α,4β,6a-α))-; Cat. No. B-6353, 6-((biotinoyl)amino)hexanoic acid, sulfosuccinimidyl ester, sodium salt, Sulfo-NHS-LC-Biotin; Cat. No. B-6352, 6-((6-((biotinoyl)amino)hexanoyl)amino)hexanoic acid, sulfosuccinimidyl ester, sodium salt, (biotin-XX, SSE); Cat. No. B-2604, biotin-X 2,4-dinitrophenyl-X-L-lysine, and DNP-X-biocytin-X.

Biotin Containing Affinity Tags for Sulfhydryls

The affinity tag Biotin-BMCC (1-biotinamide-4-[4′-(maleimidomethyl)cyclohexane-carboxamido]butane; Pierce Chemical Co., Rockford, Ill.) will form thioether bonds with sulfhydryl groups on polypeptides and other biomolecules (see, e.g., Hermanson, supra). Another affinity tag is Biotin-HPDP (N-[6-(biotinamido)hexyl]-3′-(2′-pyridyldithio)propionamide; Pierce Chemical Co.) which forms a disulfide bond with free sulfhydryls (Hermanson, supra). This bond can be disrupted through the use of reducing reagents, such as DTT (Dithiothreitol). Other useful biotin containing affinity tags that can be used to react with sulfhydryls include those affinity tags that contain an iodoacetyl or an iodacetyamidyl moiety such as iodoacetyl-LC-biotin (N-iodoacetyl-N-biotinylhexylenediamine; Pierce Chemical Co., Rockford, Ill.), and EZ-Link™ PEO-Iodoacetyl Biotin ([+]-biotinyl-iodoacetamidyl-3,6-dioxaoctanediamine; Pierce Chemical Co., Rockford, Ill. Cat. No. 21334).

Molecular Probes also provides affinity tags that contain biotin affinity labels and biomolecule reactive groups that react with sulfhydryls. Examples of these affinity tags include, without limitation, Cat. No. B-1591, N-(biotinoyl)-N′-(iodoacetyl)ethylenediamine and Nα-(3-maleimidylpropionyl)biocytin; Cat. No. M-1602.

Biotin Containing Affinity Labels for Carboxyl Groups

Other biotin containing affinity tags are contain biomolecule reactive groups that react with carbonyl or carboxyl groups, such as biotin hydrazide (cis-tetrahydro-2-oxothieno[3,4-d]-imidazoline-4-valeric acid hydrazide; Pierce Chemical Co., Rockford, Ill.), biotin-LC-hydrazide (Pierce Chemical Co., Rockford, Ill.), and biocytin hydrazide (Pierce Chemical Co., Rockford, Ill.).

Biotin Containing Photoreactive Affinity Tags

Photoreactive biomolecule reactive groups are present in certain embodiments of the present invention. Photoreactive biotin-containing affinity labels such as N-(4-azido-2-nitrophenyl)-aminopropyl-N′-(N-d-biotinyl-3-aminopropyl)-N′-methyl-1,3-propanediamine (photobiotin; Pierce Chemical Co., Rockford, Ill.) can be activated with light at 350 nm.

VII. Capture Reagents

Many substances that are useful as capture reagents have been described above (see e.g., Table 1). In certain preferred embodiments, biotin and biotin analog (e.g., iminobiotin) binding molecules are used as capture reagents. These biotin-binding capture reagents include, but are not limited to, avidin, streptavidin, NeutrAvidin biotin binding protein (Molecular Probes), Streptavidin agarose (Molecular Probes), CaptAvidin agarose (Molecular Probes), CaptAvidin biotin binding protein (Molecular Probes), Streptavidin acrylamide (Molecular Probes), or an anti-biotin antibody (e.g., mouse monoclonal 2F5 antibody (Molecular Probes).

VIII. Substrates

A substrate used in the present invention can be made of any suitable material. For example, the substrate material can include, but is not limited to, insulating materials (e.g., glass such as silicon oxide, ceramic), semi-conducting materials (e.g., silicon wafers), or electrically conducting materials (e.g., metals, such as nickel, brass, steel, aluminum, gold, or electrically conductive polymers), organic polymers, biopolymers, acrylamide, acrylamide gels, plastics, or any combinations thereof. Substrates suitable for use in embodiments of the invention are described in, e.g., U.S. Pat. No. 5,617,060 (Hutchens and Yip) and WO 98/59360.

The substrate can have various properties. The substrates generally are non-porous, e.g., solid, and substantially rigid to provide structural stability. Furthermore, the substrate can be electrically insulating or conducting. In a preferred embodiment, the substrate is electrically conducting to reduce surface charge and to improve mass resolution. Electrical conductivity can be achieved by using materials, such as electrically conductive polymers (e.g., carbonized polyetheretherketone, polyacetylenes, polyphenylenes, polypyrroles, polyanilines, polythiophenes, etc.), or conductive particulate fillers (e.g., carbon black, metallic powders, conductive polymer particulates, fiberglass-filled plastics/polymers, elastomers, etc.).

A substrate can be in any suitable shape as long as it is adapted for use with a gas phase ion spectrometer (e.g., removably insertable into a gas phase ion spectrometer). For example, the substrate can be in the form of a strip, a plate, or a dish with a series of wells at predetermined addressable locations. Typically, a substrate can take the shape of a rod, wherein a surface at one end of the rod is the sample-presenting surface, a strip or a rectangular or circular plate. Furthermore, the substrate can have a thickness of between about 0.1 mm to about 10 cm or more, preferably between about 0.5 mm to about 1 cm or more, most preferably between about 0.8 mm and about 0.5 cm or more. Preferably, the substrate itself is large enough so that it is capable being hand-held. For example; the longest cross dimension of the substrate can be at least about 1 cm or more, preferably about 2 cm or more, most preferably at least about 5 cm or more. The substrate can also be shaped for use with inlet systems and detectors of a gas phase ion spectrometer. For example, the substrate can be adapted for mounting in a horizontally and/or vertically translatable carriage that horizontally and/or vertically moves the substrate to a successive position without requiring repositioning of the substrate by hand.

In a preferred embodiment, the substrates of this invention are adapted for SELDI (Surface-Enhanced Laser Desorption/Ionization). Accordingly, the areas of the surfaces that will form the features can have adsorbents attached that will selectively bind analytes. The adsorbents can he highly specific for an analyte, such as antibodies, or they can be relatively unspecific, such as anion or cation exchange resins. Alternatively, the surface can have energy absorbing molecules or photolabile attachment groups attached. For examples of each see U.S. Pat. No. 5,719,060 (Hutchens & Yip) and WO 98/59361 (Hutchens & Yip).

The capture reagents of the present invention can be adsorbed directly or indirectly to a substrate to aid in immobilizing the affinity tagged products. The substrates can be coated with any number of different adsorbents so as long as they have binding characteristics suitable for binding capture reagents. The adsorbents can comprise a hydrophobic group, a hydrophilic group, a cationic group, an anionic group, a metal ion chelating group, lectin, heparin, or antibodies which specifically bind to markers, or a combination thereof (sometimes referred to as “a mixed mode” adsorbent). Exemplary adsorbents containing a hydrophobic group include matrices having aliphatic hydrocarbons, e.g., C₁-C₁₈ aliphatic hydrocarbons and matrices having aromatic hydrocarbon functional group such as phenyl groups. Exemplary adsorbents containing a hydrophilic group include glass (e.g., silicon oxide), or hydrophilic polymers such as polyethylene glycol, dextran, agarose, or cellulose. Exemplary adsorbents containing a cationic group include matrices of secondary, tertiary or quaternary amines. Exemplary adsorbents containing an anionic group include matrices of sulfate anions (SO₃⁻) and matrices of carboxylate anions (COO⁻) or phosphate anions (OPO₃⁻). Exemplary adsorbents containing metal chelating groups include organic molecules that have one or more electron donor groups which form coordinate covalent bonds with metal ions, such as copper, nickel, cobalt, zinc, iron, and other metal ions such as aluminum and calcium. Adsorbents that contain protein G or protein A can be used to bind to capture reagents that contain a Fc domain.

### EXAMPLES

The following examples are offered to illustrate, but not to limit the claimed invention.

### EXAMPLE 1

Analysis of Protein Containing Samples Using a Biotin Containing Affinity Label

Part 1: Denaturation and Reduction of Sample.

Due to the specificity of iodoacetamidyl for cysteine residues (reagent is selective for free sulfhydryl groups), all disulfide biomolecule (protein, etc.) species within a complex sample can first be denatured (to expose all potential cystine coupling sites within a protein for further reaction) and reduced (to break disulfide bonds).

1. Adjust protein content of the sample to 1-10 mg/ml in 0.1M sodium phosphate buffer containing 5 mM EDTA (Sigma; E5134) as an antioxidant (pH 6.0).

2. Add reducing reagent to 100 μl of protein mixture as follows:

A) 600 μg 2-mercaptoethylamine.HCl (MEA; Pierce Chemical Co., Product No. 20408). Final MEA concentration of 50 mM;

B) Dithiothreitol (DTT; Pierce Chemical Co., Product No. 20290) to a final concentration of 10 mM; or

C) Guanidine HCl (6M; Sigma, G7153) and tributyl phosphine (5 mM; Fluka, 90827) in Tris buffer (50 mM; pH 8.5).

3. Incubate for 90 min at 37° C.

4. Allow cooling to room temperature, then remove excessive reducing agent by desalting on a desalting column (Pierce Chemical Co., Product no. 43230 or 43231), into Tris buffer (50 mM, pH 8.3) containing 5 mM EDTA.

Once the reduction of the protein mixture is complete, it is necessary to remove the reducing agent (e.g., through dialysis), which would otherwise interfere with subsequent steps in the protocol. As an alternative to the reduction and desalting step mentioned above, see Example 2 as indicated below.

Part 2: Coupling of Biotinylated tag to free sulfhydryl groups.

The labeling of free sulfhydryl groups is completed according to the standard protocol supplied with the biotinylation reagent, EZ-Link™ PEO-Iodoacetyl Biotin ([+]-biotinyl-iodoacetamidyl-3,6-dioxaoctanediamine; Pierce Chemical Co., Rockford, Ill. Cat. No. 21334).

1. Add 15 ul of PEO-Iodoacetyl biotin solution to 1 ml of the reduced protein mixture from Part 1.

2. Mix and incubate in the dark for 90 minutes at room temperature.

3. Remove excessive biotinylation tag substrate from the biotinylated protein/peptide mixture by desalting on a desalting column (Pierce Chemical Co., Cat. No. 43230 or 43231), into ammonium carbonate buffer (50 mM, pH 8.3) containing 0.1% sodium dodecyl sulfate (SDS; Sigma, Cat. No. L6026).

Once the biotinylation reaction is complete, it is necessary to remove any unreacted PEO-Iodoacetyl biotin (e.g., through dialysis), which would otherwise interfere with subsequent steps in the protocol. As an alternative to the desalting step mentioned above, see Example 3, as indicated below.

Part 3: Digestion of Biotinylated Protein/Peptides.

1. To the biotinylated protein/peptides, add sequencing grade modified trypsin (Promega Corporation, Madison, Wis. Cat No. V5111). Trypsin should be resuspended in 50 mM acetic acid. For incubation of trypsin with the biotinylated protein/peptides, 5 μg of trypsin should be incubated with 100 μg of biotinylated protein/peptide for 18 hrs at 37° C.

Part 4: Capture of Biotinylated Peptide Fragments onto a Streptavidin ProteinChip® Array.

1. Preparation of ProteinChip® Array with Streptavidin as the Capture Reagent (Sigma; S4762)

A. Place an 8 spot PS2 ProteinChip® Array (Ciphergen Biosystems, Inc., Fremont, Calif.) on a flat, clean surface.

B. Apply 1 μl PBS (50 mM; pH 7.2) followed by 1 μl streptavidin (0.1 mg/ml protein concentration dissolved in 50 mM PBS, pH 7.2) to one or more spots on the 8 spot array.

C. Immediately transfer the chip to a humidity chamber (at >95% relative humidity) and incubate for 1-2 hours at room temperature or at 4° C. overnight.

D. Block residual active sites on the chip by placing the whole array in a 15 ml conical tube with 8 ml blocking buffer (0.5M Ethanolamine (Sigma; E9508); in 50 mM PBS, pH 8.0). Mix gently on a rocking platform for 15 minutes at room temperature. Note: The blocking is performed in bulk here for convenience but can also be accomplished by applying 5 μl 0.5 M Ethanolamine per spot.

E. Pour off the blocking buffer and add 8 ml PBS +0.5% Triton X-100 (Sigma; Cat. No. T9284), then agitate vigorously on a shaker for 15 minutes. Pour off the wash buffer and repeat.

2. Capture and Purification of Biotinylated Peptides

F. Dilute the biotinylated peptide preparation into a stock PBS solution containing 5% Triton X-100 to a final Triton X-100 concentration of 0.5%.

G. Pour off the wash buffer from Step E and add 5 μl of the digested, biotinylated peptide mixture to each of the ProteinChip® Array spots previously coupled with streptavidin. Incubate at high humidity for 2 hours at 4° C.

H. Remove and discard the excess biotinylated peptide mixture. Add 10 μl of PBS containing 0.5% Triton X-100. Remove this washing buffer and repeat 3 times more. Finally, wash two times with 10 μl PBS only, followed by two washes with HPLC grade water.

I. Remove the ProteinChip® array from the tube, flick off any water and allow to air dry.

J. Carefully outline the spots with a Mini PAP pen (Zymed Laboratories, Inc., San Francisco, Calif. Cat. No. 00-8877) and allow to air dry. The Mini PAP pen provides a water repellent hydrophobic barrier around the spot.

3. Addition of CHCA

A. Dissolve one tube 5 mg of alpha-cyano-7-hydroxy cinnamic acid (CHCA; Ciphergen Biosystems, Inc.) powder by adding 250 μl of 50% acetonitrile (Aldrich) containing 0.5% trifluoroacetic acid (Sigma, Cat. No. T0274).

B. Add 0.5 μl CHCA to each spot, taking care to make sure that the solution does not flow outside the spot area.

C. Dry the chip and insert it into the ProteinChip® Reader (Ciphergen Biosystems, Inc.). Collect 50-100 averaged laser shots per spot using low laser intensity.

### EXAMPLE 2

Modification 1 of Example 1

The elimination of steps involving excess sample preparation can result in higher yields of modified peptides/proteins for subsequent steps. For example, the addition of common reducing agents such as mercaptoethylamine-HCl (MEA) or dithiothreitol (DTT), as in Example 1, can interfere with subsequent reactions involving linking sulfhydryl reactive protein groups of the affinity tags to free sulfhydryls and must be removed by dialysis or buffer exchange columns. As an alternative to the use of MEA or DTT in the reduction step, the following reduction step based on solid-phase reduction can be carried out.

Materials:

Reduce-Imm™ Reducing Kit (Cat. No. 77700; Pierce Chemical Co., Rockford, Ill. 61105)

Equilibration Buffer 1: 0.1M sodium phosphate, 5 mM EDTA, pH 8.0

Equilibration Buffer 2: 0.1M sodium phosphate, 6M guanidine, 5 mM EDTA, pH 8.0

Reducing/regeneration Buffer: 0.1M sodium phosphate, 10 mM dithiothreitol, 5 mM EDTA, pH 8.0

Protocol:

All of the steps are performed at room temperature. Dilute peptide/protein sample with a solution to such that the diluted solution contains 0.1M sodium phosphate, 6M guanidine, 5 mM EDTA, pH 8.0.

Solid-phase Substrate Activation:

1. Take apart the column, which contains the solid-phase reduction substrate, supplied in the Reduce-Imm™ Reducing kit to liberate the solid-phase gel substrate (beads) and transfer this substrate into a 1.5 ml Eppendorf tube.

2. Equilibrate the beads by washing with 5 ml of Equilibration buffer 1.

3. Reduce and activate the beads by applying 10 ml of DTT solution.

4. Wash the beads with Equilibration Buffer 1 to remove free DTT with several washes (20 ml) of Equilibration buffer 2.

5. Apply peptide/protein mixture to be reduced.

6. Incubate at room temperature for 60 minutes with constant shaking.

7. Centrifuge the mixture and use the supernatant (reduced peptide/protein) for subsequent reactions.

### EXAMPLE 3

Modification 2 of Example 1:

After the biotinylated protein/peptide is prepared (by reacting the reduced protein/peptide with the biotinylated substrate tag at a molar ratio of 1:5), any unconsumed biotinylated substrate tag must be removed before subsequent specific capture of the biotinylated protein/peptides on-coated substrate. This was originally done through a desalting column. As an alternative, the following method is suggested. It relies on the incubation of the biotinylation reaction mixture with an excess of agarose beads coupled to cysteine containing free sulfhydryl groups. The excessive free sulfhydryl groups provided by the cystine-coupled agarose beads act to mop up any unconsumed biotinylated substrate, and can be eliminated from the sample preparation by centrifugation.

Prepare Cysteine-Agarose.

Reagents:

ImmunoPure Epoxy-Activated Agarose (Cat No. 20241; Pierce Chemical Co., Rockford, Ill. 61105).

L-Cystine (Cat No. C6195; Sigma Chemical Company).

Phosphate buffered saline (0.2M; pH 7.2).

Ethanolamine (1M, pH 8.5)

Protocol:

1. Add 1 g of Epoxy-activated Agarose to a 10 ml solution of Cystine (1M) and allow gel to swell.

2. Allow gel to react for 12-20 hrs at 37° C. with mixing.

3. Wash the gel 3-5 times with PBS.

4. Change buffer to 1M Ethanolamine, pH 8.5 and incubate 10-20 hrs at 37° C.

5. Wash the gel 3-5 times with PBS.

6. Reduce—SH groups on cysteine before use in as described in Example 1, Part 1: Denaturation and Reduction of Sample.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

The present invention provides novel materials and methods for analyzing biomolecular analytes in a sample. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLE 4

Comparison of the Relative amount of Horseradish Peroxidase in three samples.

In two sets of experiments, differing amounts of Horseradish Peroxidase were added to samples containing the sample amount of other proteins: Bovine IgG, Chicken Conalbumin, Bovine Serum Albumin, Horse Radish Peroxidase, and Superoxide Dismutase. The samples were then reacted with an affinity tag, PEO-Iodoacetyl Biotin and digested with trypsin. The affinity tagged products were then immobilized on streptavidin coated PS2 ProteinChips® and analyzed using mass spectrometry. A detailed description of these experiments is as follows.

### EXAMPLE 4A

Model System for Detecting Horseradish Peroxidase in three Samples.

Reagents:

Sodium Phosphate Buffer (0.1M, 5 mM EDTA; pH 6.0)

PBS (Sigma P4417)

EDTA (Sigma E5134, 0.372 g/200 ml)

Sodium Phosphate Buffer (0.1M, pH 7.5)

PBS (Sigma P4417, 1 tablet per 200 ml water)

Tris Buffer (50 mM, 5 mM EDTA; pH 8.5)

TRISma Base (Sigma T1503,; 2.4 g/400 ml)

EDTA (Sigma E5134; 0.744 g/400 ml)

Ammonium Bicarbonate Buffer (25 mM, 5 mM EDTA; pH 8.3)

Ammonium Bicarbonate (Sigma A6141,; 790.6 mg/400 ml)

EDTA (Sigma E5134, 0.744 g/400 ml)

Guanidine-HCl (6M Sigma G7153).

Tributyl Phosphine (5 mM; Fluka 90827, 3 μl into 97 μl Tris buffer (50 mM, 5 mM EDTA; pH 8.5)

PEO-Iodoacetyl Biotin (Pierce 21334; 50 mg; Fwt=542.4)

Prepare a 4 mM solution in sodium phosphate buffer (0.1 M, 5 mM EDTA; pH 6) by adding 216.8 μg PEO-Iodoacetyl Biotin to 100 μl buffer.

Trypsin, Sequencing grade, modified (Promega V511A)

Add 100 μl of supplied acetic acid buffer and use immediately.

Ethanolamine (Sigma E9508)

1 M solution in sodium phosphate (0.01 M, pH 7.5) by adding 3.05 ml ethanolamine into a final volume of 50 ml sodium phosphate buffer pH to 8.0 with concentrated HCl.

Triton-X-100 (Sigma T9284)

Prepare a 10% stock solution in deionized water.

Protein Standards (all supplied by Ciphergen; each dissolved into 100 μl Tris buffer (50 mM, containing 5 mM EDTA).

Bovine IgG (147.3 kDa; 5 nmol)

Chicken Conalbumin (77.49 kDa; 2 nmol)

Bovine Serum Albumin (66.43 kDa; 2 nmol)

Horse Radish Peroxidase (43.24 kDa; 6 nmol)

Superoxide Dismutase (15.59 kDa; 1 nmol)

Microcon Centrifugal Filters YM-3 (Amicon Bioseparations)

Reduction and Denattration of Samples Containing Proteins

1. Protein reagents were prepared as follows. Add together BSA (Bovine Serum Albumin) (100 μl), SOD (Superoxide Dismutase) (100 μl), Conalbumin (100 μl) and IgG (100 μl) and mix well. Total volume=400 μl.

2. Three reaction vials (1.5 ml Eppendorf tubes) were prepared containing the following:

TABLE 2

| Reagent | Reaction Tube A | Reaction Tube B | Reaction Tube C |
|---|---|---|---|
| Protein Mixture from | 130 μl | 130 μl | 130 μl |
| Step 1 | | | |
| Horse Radish | 50 μl | 100 μl | 150 μl |
| Peroxidase Protein Std | | | |
| Tris.EDTA buffer | 50 μl | 50 μl | 0 μl |
| Guanidine HCl | 160 mg | 160 mg | 160 mg |
| Tributyl Phosphine | 10 μl | 10 μl | 10 μl |

3. The contents of each tube were mixed well and incubated for 90 min at 37° C., and then allowed to cool to room temperature.

1. The contents of each reaction tube were transferred to a Microcon centrifugal filter (YM-3) and centrifuged at 10,000×g until the volume was reduced to approximately 50 μl. The centrifugation step was repeated 3 times by replenishing the filter with 400 μl sodium phosphate buffer (0.1 M, 5 mM EDTA; pH 6.0). The eluant was discarded after each wash.

5. The retentate was transferred to a clean reaction tube and 50 μl sodium phosphate buffer (0.1M, 5 mM EDTA; pH 6.0) was added to bring the final volume to approximately 100 μl.

Biotinylation—Generation of Affinity Tagged Products

6. 15 μl of PEO-Iodoacetyl Biotin was added to each of the reaction tubes.

7. The reaction tubes were mixed well and incubated in the dark for 90 min at room temperature. After incubation, 300 μl ammonium bicarbonate buffer was added (25 mM; 5 mM EDTA, pH 8.3).

8. The contents of each reaction tube were transferred to a Microcon centrifugal filter (YM-3) and centrifuged at 10,000×g until the volume was reduced to approximately 50 μl. The centrifugation step was repeated 3 times by replenishing the filter with 400 μl ammonium bicarbonate buffer. The eluant was discarded after each wash.

9. The retentate was transferred to a clean reaction tube.

Digestion of Protein Mixture with Trypsin

10. Trypsin (25 μl) was added to each of the reaction tubes and incubated overnight at 37° C. After incubation overnight, the reaction tubes were stored immediately at −20° C. until the time of further analysis.

Preparation of PS2 ProteinChip® Arrays

11. The streptavidin stock (5 mg/ml) was diluted 1:10 in PBS (0.1M, pH 7.5).

12. 2 μl streptavadin was applied to the spots on an 8-spot PS2 ProteinChip® Array (Ciphergen Biosystems) as indicated in Table 3 and incubated at high humidity for 1 hr at room temperature.

13. After incubation, 1 μl ethanolamine (1M, pH 8) was applied directly into the streptavidin droplet and incubated an additional 30 min.

14. Each spot of the ProteinChip® Array was washed three times with 5 μl phosphate buffer (0.01M, pH 7.5) containing 0.5% Triton-X-100. The spots were washed a final time with 5 μl phosphate buffer containing 0.05% Triton-X-100.

15. Prepare ProteinChip® Array aaac0739, aaac 1182, aaac1203 as follows:

TABLE 3

| Spot No. | streptavidin | Reaction Mixture |
|---|---|---|
| A | + | A |
| B | + | B |
| C | + | C |
| D | | |
| E | − | A |
| F | − | B |
| G | − | C |
| H | | |

16. Add CHCA to dried spots and analyze using mass spectrometry as set out in next step below.

Mass Spectrometry

All of the samples were analyzed using a ProteinChip® System-II (PBSII, Ciphergen Biosystems, Inc.). The instrument settings were as follows:

Acquisition:

High mass: 20000 Da

Spot #: A

Digitizer rate: 250.0 MHz

Ion mode: Positive

Chamber vacuum: 3.209e-007 Torr

Source voltage: 20000.0 V

Detector voltage: 1900 V

Time lag focusing: On

Pulse voltage: 3000 V

Pulse lag time: 528 ns

Focus mass: 5000.00 Da

Statistics:

Shots:

Fired: 91 Kept: 65 High: 0 Low: 0

Intensity:

Low: 259 High: 264

Sensitivity:

Low: 10 High: 10

Set high mass to 20000 Daltons, optimized from 2000 Daltons to 10000 Daltons.

Set starting laser intensity to 259.

Set starting detector sensitivity to 10.

Focus mass at 5000 Daltons.

Set data acquisition method to SELDI Quantitation

Set SELDI acquisition parameters 20. delta to 5. transients per to 5 ending position to 80.

Set warming positions with 2 shots at intensity 264 and Don't include warming shots.

Process sample.

Identify peaks using auto identify from 2000 Daltons to 10000 Daltons.

Data Analysis

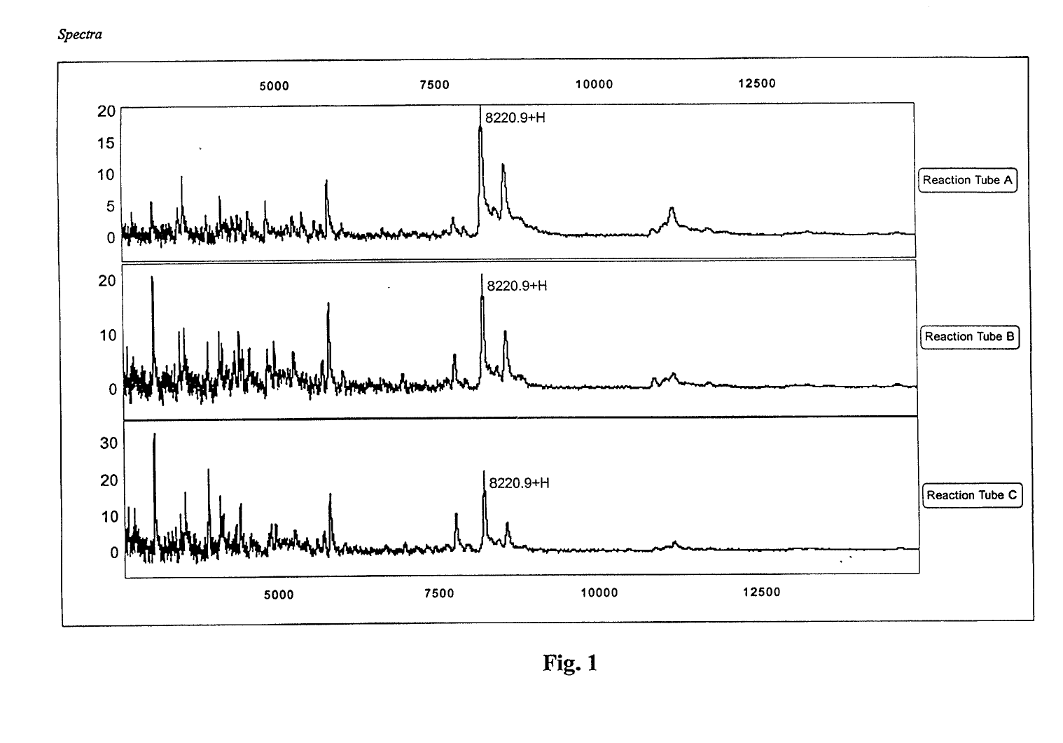

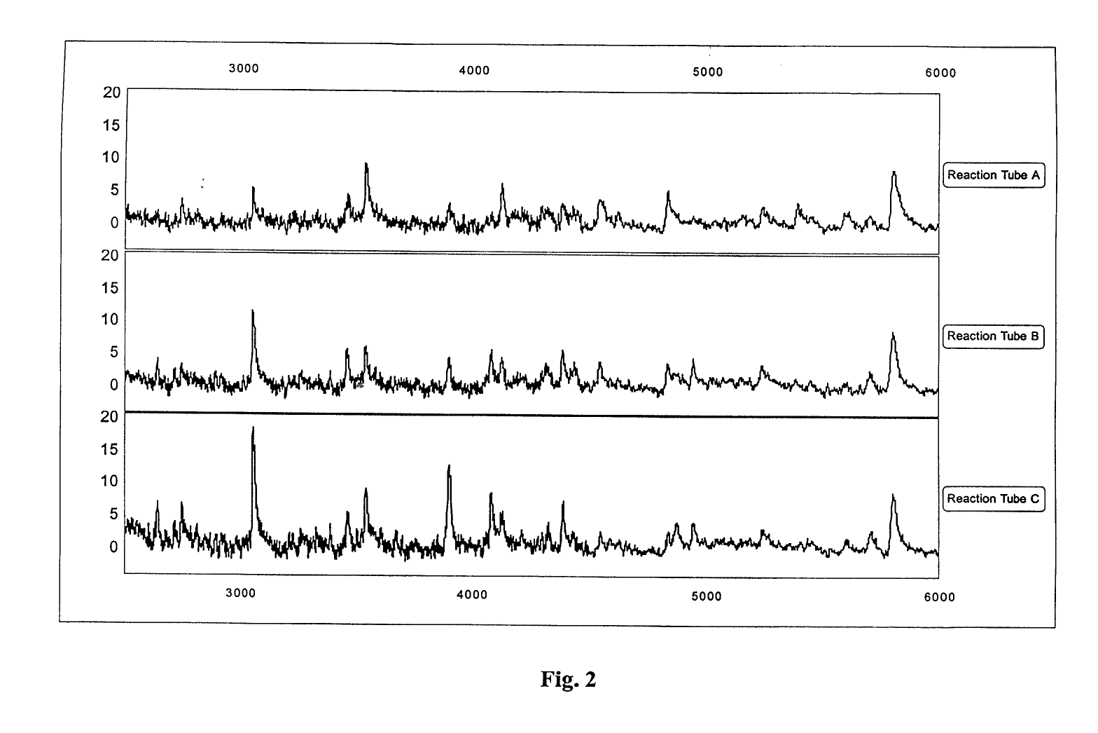

Mass spectra were generated for Reactions A, B, and C on streptavidin coated PS2 ProteinChips® (see FIGS. 1 and 2). An examination of the low mass range using the Biomarker Wizard program (Ciphergen Biosystems, Inc., Fremont, Calif.) three masses consistent with being fragments of horseradish peroxidase were identified, due in part to the increasing amount of horseradish peroxidase that was present in the original samples (e.g., 1 nmol, 2 nmol, and 3 nmol) (see arrows in FIG. 2).

The purpose of Biomarker Wizard is to generate consistent peak sets across multiple spectra. When comparing a given protein peak across various sample conditions, it is important to obtain an intensity value for each spectrum, even though they may not have been found with a given set of automatic peak detection settings. The Biomarker Wizard operates in two passes. The first pass uses low sensitivity to detect obvious and well-defined peaks. The second pass uses higher sensitivity settings to search for smaller peaks, with mass values found in the first pass. The first pass of the Biomarker Wizard operates by searching for peaks using the lowest sensitivity settings, as determined in the Automatic Peak Detection options dialog. The second pass of the Biomarker Wizard tries to complete the peak sets by using the high sensitivity values for peak detection.

The Biomarker Wizard also permits the visualization of the intensity value of a spectrum at a given mass. For example, a difference in a protein's concentration in two different samples results in a visible difference in overall intensities between the spectra for the two samples. The Biomarker Wizard also permits exporting data for further analysis in programs such as Microsoft Excel (Microsoft, Inc., Redmond, Wash.). The Biomarker Wizard can provide parameters such as output intensity and M/Z data for each peak in the mass spectrum.

Using the Biomarker Wizard, data for the three horseradish fragments identified in FIG. 2 was exported to Microsoft Excel and the average intensity (+/−CV) for each peak is displayed versus amount of horseradish peroxidase initially added to reaction in Tables 4:

TABLE 4

| | 1 nmol | | 2 nmol | | 3 nmol | |
|---|---|---|---|---|---|---|
| Mass (Da) | Avg | +/− CV | Avg | +/− CV | Avg | +/− CV |
| 3001.4 | 6.07 | 0.22 | 10.66 | 0.68 | 22.12 | 1.57 |
| 3843 | 3.37 | 0.13 | 4.56 | 0.21 | 14.06 | 0.68 |
| 4325.2 | 4.41 | 0.58 | 5.55 | 0.31 | 11.08 | 1.10 |

### EXAMPLE 4B

Model system of Horseradish Peroxidase spiked into a simple protein mixture comprising Bovine IgG, Chicken Conalbumin, Bovine Serum Albumin and Superoxide Dismutase.

Materials and Methods:

Model System Components:

1. Standard Protein Mix Preparation

As a standard protein mix, add together superoxide dismutase (1 nmol), bovine serum albumin (2 nmol), chicken conalbumin (2 nmol), and bovine IgG (5 nmol) in a final volume of 400 ul TRIS buffer (50 mM pH 8.5 containing 5 mM EDTA).

2. Spiked Protein Preparation

Horseradish peroxidase was used as a protein that could be spiked into the standard protein mix at variable concentrations. A stock solution of horseradish peroxidase was prepared by dissolving 6 nmol protein in 300 ul TRIS buffer (50 mM pH 8.5 containing 5 mM EDTA).

Reduction and Denaturation:

3. Mix together protein reagents from above according to the following table 1. Guanidine HCl was prepared by adding pre-weighed reagent directly to the reaction tube to give a final molarity of 6M. A 5 mM stock solution of Tributyl Phosphine was prepared by adding 3 ul neat Tributyl Phosphine into 97 ul TRIS buffer (50 mM, 5 mM EDTA; pH 8.5).

TABLE 5
Reaction mixture composition for Procedure.

| Reagent | Tube 0 | Tube 1 | Tube 2 | TUBE 3 |
|---|---|---|---|---|
| Standard Protein Mix | 130 ul | 130 ul | 130 ul | 0 ul |
| Horseradish peroxidase | 0 ul | 75 ul | 115 ul | 75 ul |
| TRIS (50 mM, 5 mM EDTA, pH | 160 ul | 160 ul | 160 ul | 160 ul |
| 8.5) | | | | |
| Guanidine HCl | 160 mg | 160 mg | 160 mg | 160 mg |
| Tributyl Phosphine | 10 ul | 10 ul | 10 ul | 10 ul |

4. Mix all tubes by vortexing and incubate at 37° C. for 90 min.

5. After incubation, transfer the contents of each tube to a molecular weight cutoff centrifugal filter (Microcon YM-10) and centrifuge at 9000×g until the volume is reduced to approx 50 ul. Repeat this step 3 times by adding 400 ul PBS (0.1M, 5 mM EDTA, pH 6) to fully remove the Guanidine HCl and Tributyl Phosphine. Transfer retentate (approx 50 ul) to a fresh microfuge tube and add 50 ul PBS (0.1M, 5 mM EDTA, pH 6).

Biotinylation:

6. Add 15 ul PEO-Iodoacetyl Biotin (Pierce Cat No. 21334; 0.1M in PBS pH 6 containing 5 mM EDTA) to each microfuge tube and mix by vortexing. Incubate for 90 min at room temperature and in the dark.

7. After incubation, add 300 ul ammonium bicarbonate buffer (25 mM, 5 mM EDTA, pH 8.3) and mix well by vortexing.

8. Transfer contents to a molecular weight cutoff centrifugal filter (Microcon; YM-10) and centrifuge at 9500×g until the volume is reduced to approx 50 ul. Add fresh ammonium bicarbonate buffer and repeat 3 times.

9. Transfer retentate (approx 50 ul) to a fresh microfuge tube and add 50 ul ammonium bicarbonate buffer (25 mM, 5 mM EDTA, pH 6).

Trypsin digestion:

10. To each microfuge tube add 5 ul modified Trypsin (prepared fresh by adding 100 ul of 10 mM Acetic acid to 20 ug lyophilized powder [Promega]).

11. Sample was then incubated overnight at 37° C.

12. After overnight digestion, each sample was frozen at −20° C. and stored until analysis.

Capture on Streptavadin based ProteinChip™ Arrays:

13. A Streptavadin stock solution of 5 mg/ml was prepared by diluting lyophilized streptavadin (Sigma, S-4762) powder in PBS (0.1M, pH 7.5).

14. Streptavadin-coated ProteinChip Arrays were prepared by adding 2 ul of stock streptavadin solution to spots of a Preactivated ProteinChip Array PS2 and incubating overnight at 4° C.

15. After streptavadin coupling, the surfaces were blocked with 10 ul ethanolamine (1M, pH 8) and incubated for 30° min at room temperature.

16. After bulk washing, the surfaces were washed in bulk with PBS (0.01M, pH 7.5, 0.5% triton X-100) a total of three times, 5 min each wash.

17. Two final washes were then completed using only PBS (0.01M, pH 7.5), 5 min each wash.

18. Peptide mixture prepared in tubes 0-3 were thawed on ice. Aliquots of each preparation were added to the streptavadin-ProteinChip Array according to table 6 and table 7.

TABLE 6
Preparation of streptavadin-coated PS2 arrays for peptide reporter
detection.

| Spot Code | Streptavadin | Sample Added | HRP |
|---|---|---|---|
| A | + | Tube 0 (8 ul) | 0 |
| B | + | Tube 1 (8 ul) | 40 pmol |
| C | + | Tube 2 (8 ul) | 61 pmol |
| D | + | Tube 3 (8 ul) | 40 pmol |
| E | − | Tube 0 (8 ul) | 0 |
| F | − | Tube 1 (8 ul) | 40 pmol |
| G | − | Tube 2 (8 ul) | 61 pmol |
| H | − | Tube 3 (8 ul) | 40 pmol |

TABLE 7
Preparation of streptavadin-coated PS2 arrays for reproducibility studies.

| Spot Code | Streptavadin | Sample Added | HRP |
|---|---|---|---|
| A | + | Tube 1 (8 ul) | 40 pmol |
| B | + | Tube 2 (8 ul) | 61 pmol |
| C | + | Tube 1 (8 ul) | 40 pmol |
| D | + | Tube 2 (8 ul) | 61 pmol |
| B | + | Tube 1 (8 ul) | 40 pmol |
| F | + | Tube 2 (8 ul) | 61 pmol |
| G | + | Tube 1 (8 ul) | 40 pmol |
| H | + | Tube 2 (8 ul) | 61 pmol |

19. After sample addition, the arrays were incubated for 2 hrs at room temperature and at high humidity.

20. After incubation, the array spots were washed with PBS (0.01M, pH 7.5, 0.5% triton X-100) three times, 5 min each time.

21. The array spots were then washed 2 times, 5 min each wash with PBS (0.01M, pH 7.5), followed by a quick water rinse.

22. α-cyano-7-hydroxycinaminic acid (CHCA) was prepared to a final composition of a 20% saturated CHCA solution in 50% acetonitrile (MeCN) and containing 0.1% trifluoroacetic acid (TFA). 0.5 ul of CHCA preparation was added to each spot and allowed to dry.

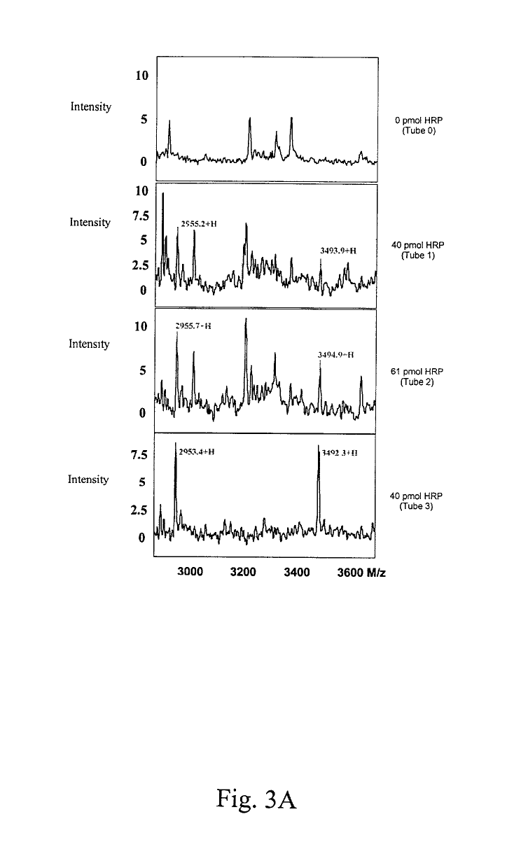

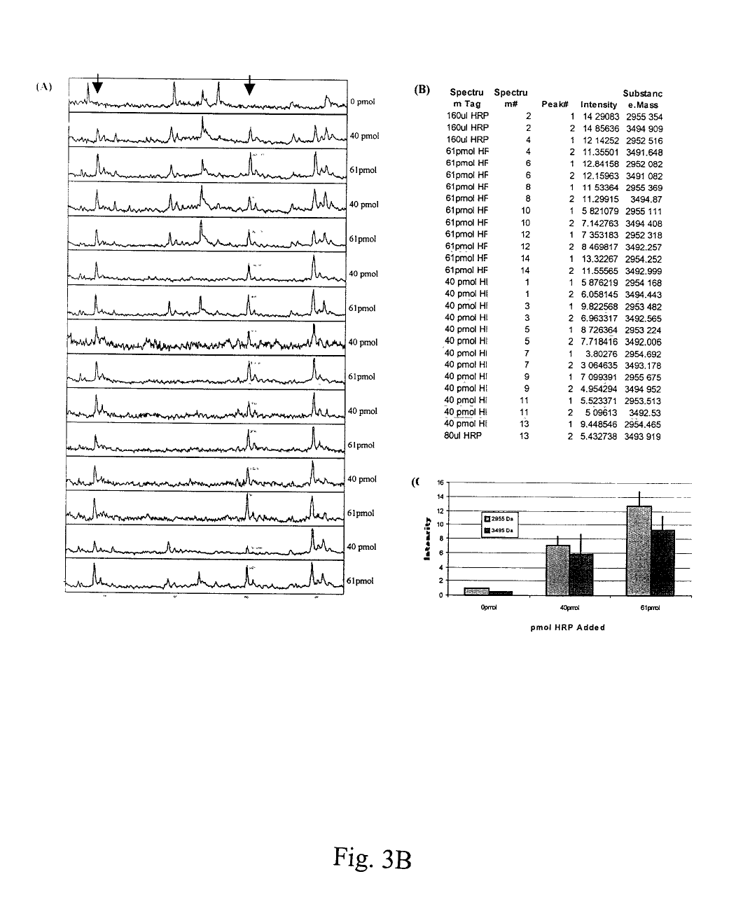

23. The Arrays were then read in a PBSII Mass Reader and the results provided in FIGS. 3A and 3B.

### Procedure Example #5

Model system of Horseradish Peroxidase spiked into a complex protein mixture.

Materials and Methods:

Model System Components:

1. Serum Preparation

Human serum was depleted of albumin using Cibacron blue dye resin (Sigma) according to manufactures recommendations. Depleted serum was finally diluted 1:1 in TRIS buffer (50 mM pH 8.5 containing 5 mM EDTA).

2. Spiked Protein Preparation

Horseradish peroxidase was used as a protein that could be spiked into the standard protein mix at variable concentrations. A stock solution of horseradish peroxidase was prepared by dissolving 6 nmol protein in 300 ul TRIS buffer (50 mM pH 8.5 containing 5 mM EDTA).

Reduction and Denaturation:

3. Mix together reaction components from above according to the following table 8. Guanidine HCl was prepared by adding pre-weighed reagent directly to the reaction tube to give a final molarity of 6M. A 5 mM stock solution of Tributyl Phosphine was prepared by adding 3 ul neat Tributyl Phosphine into 97 ul TRIS buffer (50 mM, 5 mM EDTA; pH 8.5).

TABLE 8
Reaction mixture composition for Procedure Example #2.

| Reagent | Tube 4 | Tube 5 | Tube 6 | TUBE 7 |
|---|---|---|---|---|
| Albumin depleted serum | 130 ul | 130 ul | 130 ul | 130 ul |
| Horseradish peroxidase | 0 ul | 150 ul | 150 ul | 150 ul |
| TRIS (50 mM, 5 mM EDTA, pH | 160 ul | 160 ul | 160 ul | 160 ul |
| 8.5) | | | | |
| Guanidine HCl | 160 mg | 160 mg | 160 mg | 160 mg |
| Tributyl Phosphine | 10 ul | 10 ul | 10 ul | 10 μl |

4. Mix all tubes by vortexing and incubate at 37° C. for 90 min.

5. After incubation, transfer the contents of each tube to a molecular weight cutoff centrifugal filter (Microcon YM-10) and centrifuge at 9000×g until the volume is reduced to approx 50 ul. Repeat this step 3 times by adding 400 ul PBS (0.1M, 5 mM EDTA, pH 6) to fully remove the Guanidine HCl and Tributyl Phosphine. Transfer retentate (approx 50 ul) to a fresh microfuge tube and add 50 ul PBS (0.1M, 5 mM EDTA, pH 6).

Biotinylation:

6. Add 15 ul PEO-Iodoacetyl Biotin (Pierce Cat No. 21334; 0.1M in PBS pH 6 containing 5 mM EDTA) to each microfuge tube and mix by vortexing. Incubate afor 90 min at room temperature and in the dark.

7. After incubation, add 300 ul ammonium bicarbonate buffer (25 mM, 5 mM EDTA, pH 8.3) and mix well by vortexing.

8. Transfer contents to a molecular weight cutoff centrifugal filter (Microcon; YM-10) and centrifuge at 9500×g until the volume is reduced to approx 50 ul. Add fresh ammonium bicarbonate buffer and repeat 3 times.

9. Transfer retentate (approx 50 ul) to a fresh microfuge tube and add 50 ul ammonium bicarbonate buffer (25 mM, 5 mM EDTA, pH 6).

Trypsin Digestion:

10. To each microfuge tube add 5 ul modified Trypsin (prepared fresh by adding 100 ul of 10 mM Acetic acid to 20 ug lyophilized powder [Promega]).

11. Sample was then incubated overnight at 37° C.

12. After overnight digestion, each sample was frozen at −20° C. and stored until analysis.

Capture on Streptavadin Based ProteinChip™ Arrays:

13. A Streptavidin stock solution of 5 mg/ml was prepared by diluting lyophilized streptavidin (Sigma, S-4762) powder in PBS (0.1M, pH 7.5).

14. Streptavidin-coated ProteinChip Arrays were prepared by adding 2 ul of stock streptavidin solution to spots of a Preactivated ProteinChip Array PS2 and incubating overnight at 4° C.

15. After streptavidin coupling, the surfaces were blocked with 10 ul ethanolamine (1M, pH 8) and incubated for 30° min at room temperature.

16. After bulk washing, the surfaces were washed in bulk with PBS (0.01M, pH 7.5, 0.5% triton X-100) a total of three times, 5 min each wash.

17. Two final washes were then completed using only PBS (0.01M, pH 7.5), 5 min each wash.

18. Peptide mixture prepared in tubes 0-3 were thawed on ice. Aliquots of each preparation were added to the streptavidin-ProteinChip Array according to table 8.

TABLE 8
Final preparation of streptavidin-coated PS2 arrays for peptide reported
detection.

| Spot Code | Streptavadin | Sample Added | HRP |
|---|---|---|---|
| A | + | Tube 4 (8 ul) | 0 |
| B | + | Tube 5 (8 ul) | 40 pmol |
| C | + | Tube 6 (8 ul) | 61 pmol |
| D | − | Tube 7 (8 ul) | 61 pmol |
| E | + | Tube 4 (8 ul) | 0 |
| F | + | Tube 5 (8 ul) | 40 pmol |
| G | + | Tube 6 (8 ul) | 61 pmol |
| H | − | Tube 7 (8 ul) | 61 pmol |

19. After sample addition, the arrays were incubated for 2 hrs at room temperature and at high humidity.

20. After incubation, the array spots were washed with PBS (0.01M, pH 7.5, 0.5% triton X-100) three times, 5 min each time.

21. The array spots were then washed 2 times, 5 min each wash with PBS (0.01M, pH 7.5), followed by a quick water rinse.

22. α-cyano-7-hydroxycinaminic acid (CHCA) was prepared to a final composition of a 20% saturated CHCA solution in 50% acetonitrile (MeCN) and containing 0.1% trifluoroacetic acid (TFA). 0.5 ul of CHCA preparation was added to each spot and allowed to dry.

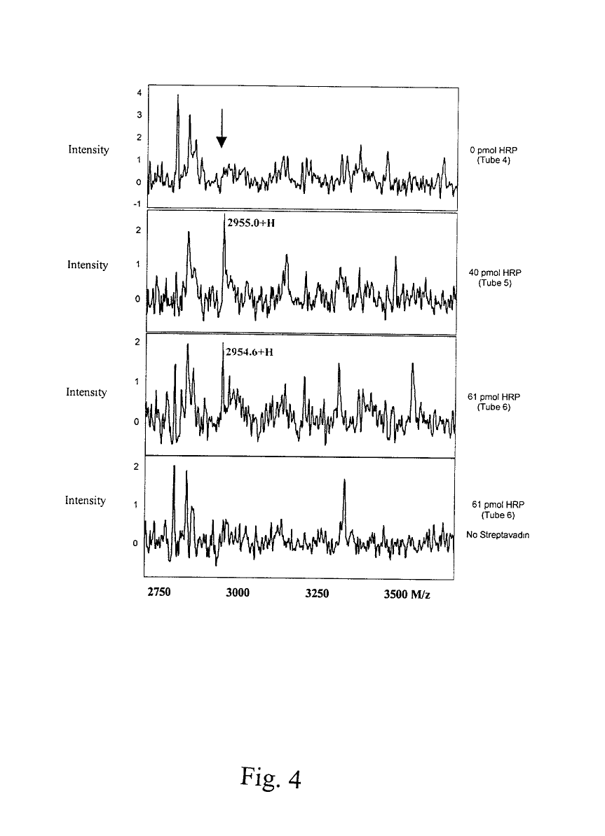

23. The Arrays were then read in a PBS-II Mass Reader and results described in FIG. 4.

What is claimed is:

1. A method for determining the relative amounts of two or more biomolecules present in a first and a second sample comprising the steps of:
(a) contacting the first and second samples in parallel with an affinity tag having the formula A—R to generate one or more affinity tagged products,
wherein the first and second samples each comprise two or more biomolecules,
wherein the biomolecule profiles of the first and the second sample overlap,
wherein A comprises an affinity label that specifically binds to a capture reagent,
wherein R comprises a biomolecule reactive group,
wherein R reacts with a functional group on the biomolecules to generate affinity tagged products comprising bonds between the affinity tag and a biomolecule;
(b) immobilizing the affinity tagged products in parallel on positionally distinguishable addresses on a substrate to generate immobilized affinity tagged products,
wherein the substrate comprises the capture reagent bound thereto;
(c) determining the amount of affinity tagged products in the immobilized affinity tagged products by mass spectrometry, wherein mass spectrometry comprises desorbing and ionizing the affinity tagged products from the immobilized affinity tagged products with an energy source and detecting the desorbed and ionized affinity tagged products with a detector; and
(d) comparing the amounts of affinity tagged products determined, whereby the comparison provides the relative amount of the biomolecules present in the first and second samples.

2. The method of claim 1, wherein said biomolecules are selected from the group consisting of: carbohydrates, lipids, proteins, nucleic acids, oligoribonucleotides, and oligodeoxyribonucleotides.

3. The method of claim 1, wherein the molecular weight of said affinity tag is less than 5000 Da.

4. The method of claim 1, wherein the molecular weight of said affinity tag is less than 1000 Da.

5. The method of claim 1, wherein the bond generated between the affinity tag and the biomolecule is a covalent bond.

6. The method of claim 1, wherein the affinity tagged products are immobilized on the substrate by contacting the affinity tagged products with a capture reagent that is bound to a substrate.

7. The method of claim 6, wherein the affinity tagged products are immobilized on the substrate by contacting the capture reagent with a substrate that binds to the capture reagent to form a capture reagent-substrate complex; and
contacting the capture reagent-substrate complex with the affinity tagged products.

8. The method of claim 1, wherein the affinity tagged products are immobilized on the substrate by contacting the affinity tagged products with a capture reagent to generate an affinity tagged product-capture reagent complex; and
contacting the affinity tagged product-capture reagent complex with a substrate that binds to the capture reagent.

9. The method of claim 1, further comprising, after step (a), contacting the affinity tagged products with a polypeptide cleaving reagent.

10. The method of claim 1, further comprising, after step (b), contacting the immobilized affinity tagged products with a polypeptide cleaving reagent.

11. The method of claim 9 or 10, wherein the polypeptide cleaving reagent is a protease.

12. The method of claim 11, wherein the protease is selected from the group consisting of: chymotrypsin, trypsin, Endoproteinase Glu-C, Endoproteinase Asp-N, Endoproteinase Lys-C, Endoproteinase Arg-C, and Endoproteinase Arg-N.

13. The method of claim 10, wherein the polypeptide cleaving reagent is cyanogen bromide or hydroxylamine.

14. The method of claim 1, wherein the first and second sample are independently selected from the group consisting of a biological sample, a blood sample, a urine sample, a cellular lysate, a tumor cell lysate, a saliva sample, a stool sample, a lymphatic fluid sample, a prostatic fluid sample, a seminal fluid sample, a milk sample, and a cell culture medium sample.

15. The method of claim 14, wherein the sample is a cellular lysate derived prepared from a cell subjected to an agent selected from the group consisting of: a chemotherapeutic agent, ultraviolet light, an exogenous gene, and a growth factor.

16. The method of claim 14, wherein the cellular lysate is selected from the group consisting of: a prokaryotic cell lysate, a plant cell lysate, a eukaryotic cell lysate, and a fungal cell lysate.

17. The method of claim 1, wherein the affinity tag further comprises a linker L to form an affinity tag having the formula A—L—R.

18. The method of claim 17, wherein L is a member selected from the group consisting of:
a C₁₋₂₀ amide, a C₁₋₂₀ polyethylene oxide, a C₁₋₂₀ polyethylene glycol, a C₁₋₂₀ polyether, a C₁₋₂₀ polyether diamine, a C₁₋₂₀ diamine, a C₁₋₂₀ polyamide, a C₁₋₂₀ polythioether, a C₁₋₂₀ silyl ether, a C₁₋₂₀ alkyl, a C₁₋₂₀ alkylenyl, and a C₁₋₂₀ alkyl-aryl group.

19. The method of claim 1, wherein the affinity tag is a member selected from the group consisting of:
biotinyl-iodoacetylamidyl-4,7,10 trioxatridecanediamine; succinimidyl D-biotin; 6-((biotinoyl)amino)hexanoic acid, succinimidyl ester; 6-((biotinoyl)amino)hexanoic acid, sulfosuccinimidyl ester; 6-((6-((biotinoyl)amino)hexanoyl) amino)hexanoic acid, sulfosuccinimidyl ester; DNP-X-biocytin-X, succinimidyl ester; (1-biotinamide-4-[4′-(maleimidomethyl)cyclohexane-carboxamido]butane; (N-[6-(biotinamido)hexyl]-3′-(2′-pyridyldithio)propionamide; N-iodoacetyl-N-biotinylhexylenediamine; [+]-biotinyl-iodoacetamidyl-3,6-dioxaoctanediamine; N-(biotinoyl)-Nα-(iodoacetyl) ethylenediamine; Nα-(3-maleimidylpropionyl)biocytin; cis-tetrahydro-2-oxothieno [3,4-d]-imidazoline-4-valeric acid hydrazide; biotin-LC-hydrazide biocytin hydrazide; and N-(4-azido-2-nitrophenyl)-aminopropyl-N′-(N-d-biotinyl-3-aminopropyl)-N′-methyl-1,3-propanediamine.

20. The method of claim 1, wherein A is a member selected from the group consisting of:
biotin, iminobiotin, glutathione, maltose, a nitrilotriacetic acid group, a polyhistidine group, an oligonucleotide, a hapten, a dinitrophenyl group, digoxigenin, a fluorophore, an Oregon Green dye, Alexa Fluor 488, fluorescein, a dansyl group, Marina Blue, tetramethylrhodamine, Texas Red, and a BODIPY dye.

21. The method of claim 1, wherein R reacts with a functional group selected from the group consisting of:
primary amines, secondary amines, hydroxyls, amines, imidazole rings, carboxylates, sulfhydryls, disulfides, thioethers, imidazolyls, phenol rings, indolyl rings, guanidinyl groups, and vicinal diols.

22. The method of claim 1, wherein R is a member selected from the group consisting of:
an activated acyl group, an activated alkyl group, a pyridyl-disulfide group, a malemide group, an iodoacetamide group, an alkyl halide, an aryl halide, a sulfonyl halide, a nitrile, an α-haloacyl group, an epoxide, an oxirane, a diazonium group, a diazoalkane, a diacetyl group, a succinimidyl ester, a N-hydroxysuccinimidyl ester, a sulfosuccinimyl ester, an isothiocyanate, an isocyanate, a sulfonyl chloride, a dichlorotriazine, an acyl azide, a pentafluorophenyl ester, a tetrafluorophenyl ester, a 4-sulfo-2,3,5,6-tetrafluorophenyl ester, a hydrazide, a 5′-(4-Fluorosulfonylbenzoyl)adenosine, and a 5-p-fluorosulfonylbenzoyl guanosine.

23. The method of claim 1, wherein the capture reagent is selected from the group consisting of protein A, avidin, steptavidin, protein G, nitrilotriacetic acid, an antibody, an anti-biotin antibody, an anti-hapten antibody, and an oligonucleotide.

24. The method of claim 1, wherein the substrate is a probe, wherein the probe comprises a surface to which the capture reagent is bound.

25. The method of claim 1, comprising, before mass spectrometry, the step of placing the immobilized affinity tagged products on a probe that is removably insertable into the mass spectrometer.

26. The method of claim 1, wherein the mass spectrometry is carried out using a laser desorption-ionization mass spectrometer.

27. The method of claim 26, wherein the laser desorption mass spectrometer is coupled to a quadrupole time-of-flight mass spectrometer.

28. The method of claim 1, wherein the mass spectrometry is carried out using a tandem mass spectrometer.

29. The method of claim 1 or 10, wherein the step of comparing comprises:
generating a first mass spectrum on the desorbed/ionized affinity tagged products of the first sample with the mass spectrometer;
generating a second mass spectrum on the desorbed/ionized affinity tagged products of the second sample with the mass spectrometer;
executing an algorithm with a programmable digital computer, wherein the algorithm:
identifies at least one peak value in the first mass spectrum and the second mass spectrum; and
compares the signal strength of the peak value of the first mass spectrum to the signal strength of the peak value of the second mass spectrum of the mass spectrum.

30. A method for determining the identity of one or more proteins in a sample comprising the steps of:
(a) contacting the sample with an affinity tag having the formula A—R to generate one or more affinity tagged products,
wherein the sample comprises one or more proteins,
wherein A comprises an affinity label that specifically binds to a capture reagent,
wherein R comprises a biomolecule reactive group,
wherein R reacts with a functional group on the biomolecules to generate affinity tagged products comprising bonds between the affinity tag and the biomolecules;
(b) immobilizing the affinity tagged products on a substrate to generate immobilized affinity tagged products,
wherein the substrate comprises the capture reagent bound thereto; and
(c) determining the identity of the protein by mass spectrometry, wherein mass spectrometry comprises desorbing and ionizing the affinity tagged products from the substrate-bound capture reagent with an energy source and detecting the desorbed and ionized affinity tagged products with a detector.

31. The method of claim 30, further comprising:
contacting said immobilized affinity tagged products with a polypeptide cleaving reagent to create polypeptide cleavage fragments; and
determining the identity of one of the proteins by analyzing at least one polypeptide cleavage fragment by mass spectrometry, wherein said mass spectrometry involves a first and second mass spectrometer, comprising
desorbing the protein cleavage fragments from the substrate-bound capture reagent to generate parent ion peptides,
selecting a parent ion peptide for subsequent fragmentation with a first mass spectrometer,
fragmenting the selected parent ion peptide under selected fragmentation conditions in the first mass spectrometer to generate product ion fragments;
generating a first mass spectrum of the product ion fragments with a second mass spectrometer; and
accessing a database with a programmable digital computer, wherein the database comprises one or more predicted mass spectra of amino acid sequences; and
executing an algorithm with a programmable digital computer, wherein the algorithm determines at least a first measure for each of the predicted mass spectra, the first measure being an indication of the closeness-of-fit between the first mass spectrum and each of the predicted mass spectra.

32. The method of claim 30, wherein the affinity tagged products are immobilized on the substrate by contacting the affinity tagged products with a capture reagent that is bound to a substrate.

33. The method of claim 32, wherein the affinity tagged products are immobilized on the substrate by contacting the capture reagent with a substrate that binds to the capture reagent to form a capture reagent-substrate complex; and
contacting the capture reagent-substrate complex with the affinity tagged products.

34. The method of claim 30, wherein the affinity tagged products are immobilized on the substrate by contacting the affinity tagged products with a capture reagent to generate an affinity tagged product-capture reagent complex; and
contacting the affinity tagged product-capture reagent complex with a substrate that binds to the capture reagent.

35. The method of claim 30, wherein the molecular weight of said affinity tag is less than 5000 Da.

36. The method of claim 30, wherein the molecular weight of said affinity tag is less than 1000 Da.

37. The method of claim 30, wherein the bond generated between the affinity tag and the biomolecule is a covalent bond.

38. The method of claim 30, further comprising, after step (a), contacting the affinity tagged products with a polypeptide cleaving reagent.

39. The method of claim 30, further comprising, after step (b), contacting the immobilized affinity tagged products with a polypeptide cleaving reagent.

40. The method of claim 30, wherein the polypeptide cleaving reagent is a protease.

41. The method of claim 40, wherein the protease is selected from the group consisting of: chymotrypsin, trypsin, Endoproteinase Glu-C, Endoproteinase Asp-N, Endoproteinase Lys-C, Endoproteinase Arg-C, and Endoproteinase Arg-N.

42. The method of claim 39, wherein the polypeptide cleaving reagent is cyanogen bromide or hydroxylamine.

43. The method of claim 30, wherein the first and second sample are independently selected from the group consisting of a biological sample, a blood sample, a urine sample, a cellular lysate, a tumor cell lysate, a saliva sample, a stool sample, a lymphatic fluid sample, a prostatic fluid sample, a seminal fluid sample, a milk sample, and a cell culture medium sample.

44. The method of claim 43, wherein the sample is a cellular lysate derived prepared from a cell subjected to an agent selected from the group consisting of: a chemotherapeutic agent, ultraviolet light, an exogenous gene, and a growth factor.

45. The method of claim 43, wherein the cellular lysate is selected from the group consisting of: a prokaryotic cell lysate, a plant cell lysate, a eukaryotic cell lysate, and a fungal cell lysate.

46. The method of claim 30, wherein the affinity tag further comprises a linker L to form an affinity tag having the formula A—L—R.

47. The method of claim 46, wherein L is a member selected from the group consisting:
a C₁₋₂₀ amide, a C₁₋₂₀ polyethylene oxide, a C₁₋₂₀ polyethylene glycol, a C₁₋₂₀ polyether, a C₁₋₂₀ polyether diamine, a C₁₋₂₀ diamine, a C₁₋₂₀ polyamide, a C₁₋₂₀ polythioether, a C₁₋₂₀ silyl ether, a C₁₋₂₀ alkyl, a C₁₋₂₀ alkylenyl, and a C₁₋₂₀ alkyl-aryl group.

48. The method of claim 30, wherein the affinity tag is a member selected from the group consisting of:
biotinyl-iodoacetylamidyl-4,7,10 trioxatridecanediamine; succinimidyl D-biotin; 6-((biotinoyl)amino)hexanoic acid, succinimidyl ester; 6-((biotinoyl)amino)hexanoic acid, sulfosuccinimidyl ester; 6-((6-((biotinoyl)amino)hexanoyl) amino)hexanoic acid, sulfosuccinimidyl ester; DNP-X-biocytin-X, succinimidyl ester; (1-biotinamide-4-[4′-(maleimidomethyl)cyclohexane-carboxamido]butane; (N-[6-(biotinamido)hexyl]-3′-(2′-pyridyldithio)propionamide; N-iodoacetyl-N-biotinylhexylenediamine; [+]-biotinyl-iodoacetamidyl-3,6-dioxaoctanediamine; N-(biotinoyl)-N′-(iodoacetyl) ethylenediamine; Nα-(3-maleimidylpropionyl)biocytin; cis-tetrahydro-2-oxothieno[3,4-d]-imidazoline-4-valeric acid hydrazide; biotin-LC-hydrazide biocytin hydrazide; and N-(4-azido-2-nitrophenyl)-aminopropyl-N′-(N-d-biotinyl-3-aminopropyl)-N′-methyl-1,3-propanediamine.

49. The method of claim 30, wherein A is a member selected from the group consisting of:
biotin, iminobiotin, glutathione, maltose, a nitrilotriacetic acid group, a polyhistidine group, an oligonucleotide, a hapten, a dinitrophenyl group, digoxigenin, a fluorophore, an Oregon Green dye, Alexa Fluor 488, fluorescein, a dansyl group, Marina Blue, tetramethylrhodamine, Texas Red, and a BODIPY dye.

50. The method of claim 30, wherein R reacts with a functional group selected from the group consisting of:
primary amines, secondary amines, hydroxyls, amines, imidazole rings, carboxylates, sulfhydryls, disulfides, thioethers, imidazolyls, phenol rings, indolyl rings, guanidinyl groups, and vicinal diols.

51. The method of claim 30, wherein R is selected from the group consisting of:
an activated acyl group, an activated alkyl group, a pyridyl-disulfide group, a malemide group, an iodoacetamide group, an alkyl halide, an aryl halide, a sulfonyl halide, a nitrile, an α-haloacyl group, an epoxide, an oxirane, a diazonium group, a diazoalkane, a diacetyl group, a succinimidyl ester, a N-hydroxysuccinimidyl ester, a sulfosuccinimyl ester, an isothiocyanate, an isocyanate, a sulfonyl chloride, a dichlorotriazine, an acyl azide, a pentafluorophenyl ester, a tetrafluorophenyl ester, a 4-sulfo-2,3,5,6-tetrafluorophenyl ester, a hydrazide, a 5′-(4-Fluorosulfonylbenzoyl)adenosine, and a 5-p-fluorosulfonylbenzoyl guanosine.

52. The method of claim 30, wherein the capture reagent is a member selected from the group consisting of protein A, avidin, steptavidin, protein G, nitrilotriacetic acid, an antibody, an anti-biotin antibody, an anti-hapten antibody and an oligonucleotide.

53. The method of claim 30, wherein the substrate is a probe, wherein the probe comprises a surface to which the capture reagent is bound.

54. The method of claim 30, comprising, before mass spectrometry, the step of placing the immobilized affinity tagged products on a probe that is removably insertable into the mass spectrometer.

55. The method of claim 30, wherein the mass spectrometry is carried out using a laser desorption-ionization mass spectrometer.

56. The method of claim 55, wherein the laser desorption mass spectrometer is coupled to a quadrupole time-of-flight mass spectrometer.

57. The method of claim 30, wherein the mass spectrometry is carried out using a tandem mass spectrometer.

58. The method of claim 30, wherein the step of determining the identity of the protein by mass spectrometry comprises:
generating a first mass spectrum on the desorbed/ionized affinity tagged components with the mass spectrometer;
accessing a database with a programmable digital computer, wherein the database comprises one or more predicted mass spectra of amino acid sequences; and
executing an algorithm with a programmable digital computer, wherein the algorithm determines at least a first measure for each of the predicted mass spectra, the first measure being an indication of the closeness-of-fit between the first mass spectrum and each of the predicted mass spectra.

59. The method of claim 38 or 39, wherein the step of comparing comprises generating a first mass spectrum on the desorbed/ionized affinity tagged components with the mass spectrometer;
accessing a database with a programmable digital computer, wherein the database comprises one or more predicted mass spectra of amino acid sequences predicted to be generated upon treatment of cleavage of the one or more proteins with the polypeptide cleaving agent; and
executing an algorithm with a programmable digital computer, wherein the algorithm determines at least a first measure for each of the predicted mass spectra, the first measure being an indication of the closeness-of-fit between the first mass spectrum and each of the predicted mass spectra.

60. A method for determining the mass of a biomolecule, comprising the steps of:
(a) contacting the biomolecule with an affinity tag having the formula A—R to generate one or more affinity tagged products,
wherein A comprises an affinity label that specifically binds to a capture reagent,
wherein R comprises a protein reactive group,
wherein R reacts with a functional group on the biomolecules to generate affinity tagged products comprising bonds between the affinity tag and the biomolecules;
(b) immobilizing the affinity tagged products on a substrate to generate immobilized affinity tagged products,
wherein the substrate comprises the capture reagent bound thereto; and
(c) determining the mass of the affinity tagged products in the immobilized affinity tagged products by mass spectrometry, wherein mass spectrometry comprises desorbing and ionizing the affinity tagged products from the immobilized affinity tagged products with an energy source and detecting the desorbed and ionized affinity tagged products with a detector.

61. The method of claim 60, wherein said biomolecule is selected from the group consisting of: a carbohydrate, a lipid, a protein, a nucleic acid, an oligoribonucleotide, and an oligodeoxyribonucleotide.

62. The method of claim 60, wherein the affinity tagged products are immobilized on the substrate by contacting the affinity tagged products with a capture reagent that is bound to a substrate.

63. The method of claim 62, wherein the affinity tagged products are immobilized on the substrate by contacting the capture reagent with a substrate that binds to the capture reagent to form a capture reagent-substrate complex; and
contacting the capture reagent-substrate complex with the affinity tagged products.

64. The method of claim 60, wherein the affinity tagged products are immobilized on the substrate by contacting the affinity tagged products with a capture reagent to generate an affinity tagged product-capture reagent complex; and
contacting the affinity tagged product-capture reagent complex with a substrate that binds to the capture reagent.

65. The method of claim 60, wherein the bond generated between the affinity tag and the biomolecule is a covalent bond.

66. The method of claim 60, wherein the affinity tag further comprises a linker L to form an affinity tag having the formula A—L—R.

67. The method of claim 60, wherein the molecular weight of said affinity tag is less than 5000 Da.

68. The method of claim 60, wherein the molecular weight of said affinity tag is less than 1000 Da.

69. The method of claim 60, further comprising, after step (a), contacting the affinity tagged products with a polypeptide cleaving reagent.

70. The method of claim 60, further comprising, after step (b), contacting the immobilized affinity tagged products with a polypeptide cleaving reagent.

71. The method of claim 69 or 70, wherein the polypeptide cleaving reagent is a protease.

72. The method of claim 71, wherein the protease is selected from the group consisting of: chymotrypsin, trypsin, Endoproteinase Glu-C, Endoproteinase Asp-N, Endoproteinase Lys-C, Endoproteinase Arg-C, and Endoproteinase Arg-N.

73. The method of claim 70, wherein the polypeptide cleaving reagent is cyanogen bromide or hydroxylamine.

74. The method of claim 60, wherein the first and second sample are independently selected from the group consisting of a biological sample, a blood sample, a urine sample, a cellular lysate, a tumor cell lysate, a saliva sample, a stool sample, a lymphatic fluid sample, a prostatic fluid sample, a seminal fluid sample, a milk sample, and a cell culture medium sample.
